(19) United States
(12) Patent Application Publication
(10) Pub. No.: US 2015/0104358 A1
(43) Pub. Date: Apr. 16, 2015

(54) NUCLEIC ACID EXTRACTION DEVICE, NUCLEIC ACID EXTRACTION KIT, AND NUCLEIC ACID EXTRACTION APPARATUS

(71) Applicant: Seiko Epson Corporation, Tokyo (JP)

(72) Inventor: Masato HANAMURA, Shiojiri (JP)

(21) Appl. No.: 14/510,359

(22) Filed: Oct. 9, 2014

(30) Foreign Application Priority Data
Oct. 11, 2013 (JP) 2013-213466

(51) Int. Cl. C12N 15/10; B01L 3/00

(52) U.S. Cl. CPC C12N 15/1013; B01L 3/502761; B01L 3/502784; B01L 2300/161; B01L 2300/0838; B01L 2200/0631; B01L 2200/0673; B01L 2200/0668; B01L 2400/043
USPC 422/527

(57) ABSTRACT

A nucleic acid extraction device has a tube in which a first plug formed of an oil, a second plug formed of a washing liquid which does not mix with an oil, a third plug formed of an oil, a fourth plug formed of an eluate which does not mix with an oil, and a fifth plug formed of an oil are arranged in this order, and the tube is subjected to an antistatic treatment.

### BACKGROUND

1. Technical Field

The present invention relates to a nucleic acid extraction device, a nucleic acid extraction kit, and a nucleic acid extraction apparatus.

2. Related Art

In recent years, medical care using genes, such as gene diagnosis and gene therapy, has attracted attention due to the development of techniques for using genes. In the agriculture and stockbreeding field, many methods using genes have also been developed for variety determination and breeding. A technique such as polymerase chain reaction (PCR) is widely used as a technique for using genes. Nowadays, the PCR is an essential technique in analyzing the information of a biological substance. The PCR is a method of applying a thermal cycle to a solution (reaction liquid) containing a nucleic acid (target nucleic acid) which is an amplification object and a reagent to amplify the target nucleic acid. As for the thermal cycle of the PCR, a method of applying a thermal cycle at two- or three-stage temperatures is generally used.

Currently, a simple examination kit such as an immunochromatography kit is mainly used for diagnosis of infections represented by influenza in the medical practice. However, in such a simple examination, the accuracy thereof may be insufficient, and PCR with which higher examination accuracy can be expected is desired to be applied to the diagnosis of infections. In general outpatient practices and the like in medical institutions, the time which can be spent for examination is limited to a short time since the time for diagnosis is limited. Therefore, for example, identification of influenza has been performed at the sacrifice of examination accuracy to reduce the time with simple examination such as immunochromatography.

Due to such a circumstance, it is necessary to reduce the time required for reaction in order to realize the examination by PCR with which higher accuracy can be expected. For example, JP-A-2009-136250 discloses, as an apparatus for causing a PCR in a short time, a biological sample reactor in which a chip for a biological sample reaction filled with a reaction liquid and a liquid which does not mix with the reaction liquid and has a lower specific gravity than the reaction liquid is rotated around a horizontal rotary shaft to move the reaction liquid, thereby applying a thermal cycle (JP-A-2009-136250). In addition, a method using magnetic beads (JP-A-2009-207459), a method using magnetic beads as a droplet moving section to move droplets in a temperature changeable area on a substrate, thereby applying a thermal cycle of PCR (JP-A-2008-012490), and the like are disclosed as one method for PCR.

Although reducing the time required for the thermal cycle of PCR has been considered, a technique of reducing the time required to extract a nucleic acid serving as a template from a sample and to thus provide a state in which the PCR can be started is not known to have been sufficiently developed. For example, a process of extracting a nucleic acid (DNA: Deoxyribonucleic Acid, and/or RNA: Ribonucleic Acid) serving as a template from a sample (blood, nasal cavity mucous, oral mucous membrane, and the like) (hereinafter, may be simply referred to as “preprocessing”) is needed to perform the PCR, but even when only the time required for thermal cycle of the PCR can be reduced, it is not possible to sufficiently deal with the request from the medical practice when the time required to extract the nucleic acid (preprocessing) cannot be reduced.

Usually, although preprocessing using a column or magnetic beads is performed, reagent dispensing, stirring, a centrifugal process, and the like are all manually performed, or an apparatus which is expensive and large-scale such as an automatic extraction apparatus is needed. In addition, in any method, preprocessing takes at least 30 minutes and great effort. Accordingly, even though only the thermal cycle of the PCR can be applied in a short time (for example, within 15 minutes), when the time required for preprocessing is added, the entire examination time from when a sample is collected to when an examination result is obtained is approximately at least one hour.

Accordingly, collectively performing processes from extraction of a nucleic acid (preprocessing) to a thermal cycle of PCR is practically impossible in the field having a limitation in consultation hours. Such a circumstance becomes an obstacle to diffusion of the examination method using PCR to medical institutions. That is, the time which is spent in PCR and preprocessing and the complexity thereof are causes of the difficulty in diffusion in the medical practice, regardless of the fact that the PCR is an examination method having higher sensitivity and higher accuracy than immunochromatography.

### SUMMARY

An advantage of some aspects of the invention is to provide a nucleic acid extraction device, a nucleic acid extraction kit, and a nucleic acid extraction method capable of reducing the time required for preprocessing for PCR.

A nucleic acid extraction device according to an aspect of the invention includes a tube in which a first plug formed of an oil, a second plug formed of a washing liquid which does not mix with an oil, a third plug formed of an oil, a fourth plug formed of an eluate which does not mix with an oil, and a fifth plug formed of an oil are arranged in this order, and the tube is subjected to an antistatic treatment.

The tube may be coated with an antistatic agent, an antistatic sheet may be wound on the tube, or the tube may include an antistatic material. The first plug, the third plug, or the fifth plug may contain an antistatic agent. In this case, it is preferable that the antistatic agent have a volume specific resistivity of 5.4×10¹⁰ Ω·cm or lower.

In this specification, a liquid “plug” refers to a material shaped so that only the liquid substantially occupies the inside in a longitudinal direction of a tube or a tube portion, and refers to a state in which the internal space of the tube or the tube portion is partitioned by the plug. Here, the expression “substantially” refers to a situation in which a small amount (for example, thin film shape) of another substance (liquid or the like) may exist around the plug, that is, on an internal wall of the tube or the tube portion. The tube or the tube portion refers to a tubular part. The tube or the tube portion refers to a deformable tubular part which has a cross-section with an internal cavity so that the liquid can maintain the plug state in the tube or the tube portion.

A nucleic acid extraction apparatus according to another aspect of the invention includes the nucleic acid extraction device, a magnetic force application portion which applies, when the tube is mounted on a mounting portion, a magnetic force from a side surface of the tube, and a moving mechanism which changes the relative arrangement between the mounting portion and the magnetic force application portion in a longitudinal direction of the tube.

A nucleic acid extraction kit according to still another aspect of the invention includes the nucleic acid extraction device, and a container which can be connected to an end on the side of the first plug of the tube by internal communication.

### BRIEF DESCRIPTION OF THE DRAWINGS

The invention will be described with reference to the accompanying drawings, wherein like numbers reference like elements.

FIG. 1 is a diagram schematically illustrating a main part of a nucleic acid extraction device according to an embodiment.

FIG. 2 is a diagram schematically illustrating a main part of a nucleic acid extraction device according to an embodiment.

FIG. 3 is a diagram schematically illustrating a main part of a nucleic acid extraction device according to an embodiment.

FIG. 4 is a diagram schematically illustrating a nucleic acid extraction device according to an embodiment.

FIG. 5 is a diagram schematically illustrating a nucleic acid extraction device according to an embodiment.

FIG. 6 is a diagram schematically illustrating a main part of a nucleic acid extraction device according to an embodiment.

FIG. 7 is a diagram schematically illustrating an example of a nucleic acid extraction kit according to an embodiment.

FIG. 8 is a diagram schematically illustrating an example of a nucleic acid extraction kit according to an embodiment.

FIG. 9 is a schematic diagram for illustrating a modification example of a nucleic acid extraction method of an embodiment.

FIG. 10 is a perspective view illustrating an example of a nucleic acid extraction apparatus according to an embodiment.

FIG. 11 is a perspective view illustrating an example of a nucleic acid extraction apparatus according to an embodiment.

FIG. 12 shows graphs illustrating results of experimental examples.

FIG. 13 is a graph illustrating a relation between an elution temperature and a DNA yield.

### DESCRIPTION OF EXEMPLARY EMBODIMENTS

Hereinafter, several embodiments of the invention will be described. Embodiments to be described below are provided to describe examples of the invention. The invention is not limited to the following embodiments, and includes various modifications implemented without changing the gist of the invention. The entire configuration to be described below is not necessarily an essential constituent element of the invention.

### 1. NUCLEIC ACID EXTRACTION DEVICE

A nucleic acid extraction device 1000 of this embodiment has a tube portion 100, a first plug 10, a second plug 20, a third plug 30, a fourth plug 40, and a fifth plug 50.

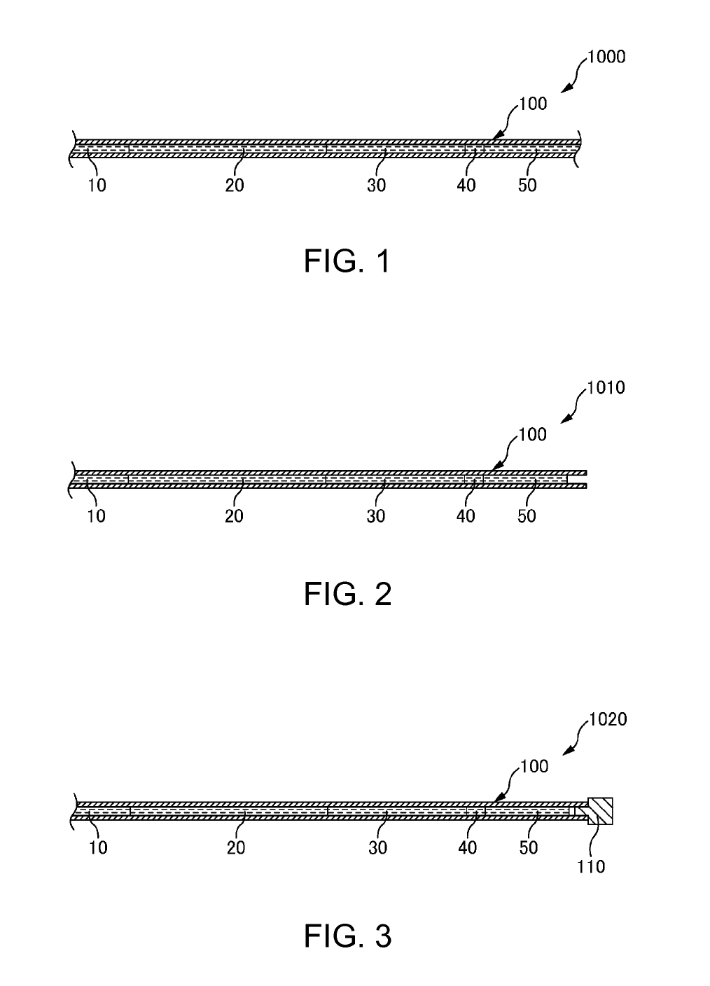

FIG. 1 is a diagram schematically illustrating a main part of the nucleic acid extraction device 1000 of this embodiment.

### 1.1. Tube Portion

The tube portion 100 constitutes a main part of the nucleic acid extraction device 1000. The nucleic acid extraction device 1000 may include various configurations other than the tube portion 100. Although not illustrated in FIG. 1, the nucleic acid extraction device 1000 may include a pipe, a container, a cock, a joint, a pump, a controller, and the like connected to the tube portion 100.

The tube portion 100 is a tubular part which has a cavity therein and can allow a liquid to flow in the cavity in a longitudinal direction. The tube portion 100 is in the longitudinal direction, but may be bent. The cavity in the tube portion 100 is not particularly limited in size and shape as long as the liquid can maintain a plug shape in the tube portion 100. The size of the internal cavity and the shape of a cross-section perpendicular to the longitudinal direction of the tube portion 100 may be changed in the longitudinal direction of the tube portion 100. Whether a liquid can maintain a plug shape in the tube portion 100 depends on conditions such as a material of the tube portion 100 and the kind of the liquid, and thus the shape of the cross-section perpendicular to the longitudinal direction of the tube portion 100 is appropriately designed within a range in which the liquid can maintain a plug shape in the tube portion 100.

The shape of the cross-section perpendicular to the longitudinal direction of the external form of the tube portion 100 is also not limited. The thickness (a length from a side surface of the internal cavity to an external surface) of the tube portion 100 is also not particularly limited. When the cross-section perpendicular to the longitudinal direction of the internal cavity of the tube portion 100 has an annular shape, the internal diameter (a diameter of the circle of the cross-section perpendicular to the longitudinal direction of the internal cavity) of the tube portion 100 can be set to, for example, 0.5 mm to 3 mm. It is preferable that the internal diameter of the tube portion 100 be within this range since a liquid plug is easily formed with a wide range of the material of the tube portion 100 and the kind of the liquid.

The material of the tube portion 100 is not particularly limited, but for example, glass, a polymer, a metal or the like can be used. However, it is preferable that a material having transparency with respect to visible light, such as glass and a polymer, be selected as the material of the tube portion 100 since the inside (the inside of the cavity) can be observed from the outside of the tube portion 100. It is preferable that a substance transmitting a magnetic force or a non-magnetic body be selected as the material of the tube portion 100 since when magnetic particles pass through the tube portion 100, it is easily performed by giving a magnetic force from the outside of the tube portion 100.

In the tube portion 100, the first plug 10 formed of an oil, the second plug 20 formed of a first washing liquid which does not mix with the oil, the third plug 30 formed of an oil which does not mix with the first washing liquid, the fourth plug 40 formed of an eluate which does not mix with the oil, and the fifth plug 50 formed of an oil which does not mix with the eluate are arranged in this order.

However, when the tube portion 100 is filled with an oil and an internal aqueous solution such as a washing liquid and an eluate, or the nucleic acid extraction device 1000 is carried or used, an electric field is generated between the aqueous solution and the tube. Thus, for example, when the aqueous solution is to be pushed out of the tube as will be described later, the aqueous solution is pulled toward and adheres to the internal wall of the tube, and only the oil is pushed out. Accordingly, the aqueous solution may not move, plug division may occur, or the aqueous solution may repel from the internal wall of the tube and droplets of the aqueous solution may float in the oil. The tube portion 100 may also be charged when a charged object such as a hand wearing a nitrile glove is moved closer to or brought into contact with the tube portion 100, and similar problems may occur. The aqueous solution divided or becoming droplets may move in the oil by the action of static electricity and mix with another plug formed of a preprocessing reagent, and thus the composition of the aqueous solution of the mixed plug may be changed and the respective plugs may lose their functions.

Therefore, the tube is preferably subjected to an antistatic treatment in the nucleic acid extraction device 1000. The method of subjecting the tube to the antistatic treatment is not particularly limited. For example, the outer surface of the tube portion 100 can be coated with the following materials: (1) organic electrolytes such as non-ionic surfactants, e.g., poly(oxyethylene)alkylamine, poly(oxyethylene)alkylamide, poly(oxyethylene)alkyl ether, poly(oxyethylene)alkylphenyl ether, glycerin fatty acid ester, and sorbitan fatty acid ester; anionic surfactants, e.g., alkylsulfonate, alkylbenzenesulfonate, alkylsulfate, and alkylphosphate; cationic surfactants, e.g., hydroxyalkyl ethanolamine, quaternary ammonium chloride, quaternary ammonium sulfate, and quaternary ammonium nitrate; and amphoteric surfactants, e.g., alkyl betaine, alkyl imidazoline, and alkyl alanine, (2) antistatic agents such as conductive metal fine particles formed of a metal such as gold, silver, copper, aluminum, iron, nickel, palladium, and platinum, or an alloy thereof, and conductive metal oxide fine particles containing, as a main component, antimony-doped tin oxide (ATO), antimony pentoxide, indium oxide-tin (ITO), zirconium oxide (zirconia), zinc oxide, ZnSbO₆, TiO₂, SnO₂, Al₂O₃, In₂O₃, SiO₂, MgO, BaO, MoO₃, V₂O₅ or the like, and lubricants such as a carbinol-modified silicone oil, a polyether-modified silicone oil, and a methylphenyl-modified silicone oil. Otherwise, a sheet coated with an antistatic agent, a sheet coated with a fluororesin, an antistatic sheet formed of a vinyl chloride film or the like, an antistatic film, or an antistatic tape may be wound thereon. An antistatic tube may be used as the tube portion 100. In that case, a commercially available tube (manufactured by CKD) may be used, but the tube may be made of an antistatic material. For example, the tube may be made of a plastic provided by kneading glycerin fatty acid ester such as monoglyceride, polyglyceryl fatty acid ester, sorbitan fatty acid ester, polyoxyethylene.sorbitan fatty acid ester, polyoxyethylene.glycerin fatty acid ester, or the like.

An antistatic agent such as a trifluoroalkyldimethyl trimethylsiloxysilicic acid may be added to the oil which fills the tube portion 100 to suppress electrification. The amount of the antistatic agent to be added is not particularly limited, but is preferably 4% or greater in the case of the trifluoroalkyldimethyl trimethylsiloxysilicic acid. In view of an antistatic effect, the antistatic agent is preferably added so that the volume specific resistivity of the oil is 5.4×10¹⁰ Ω·cm or lower. Here, the volume specific resistivity is a term which means the electric resistivity of the material, and is also called volume resistivity.

In this manner, when the electrification of the nucleic acid extraction device 1000 is prevented, the positions of the respective plugs in the tube portion 100 can be stably maintained. When the position of the aqueous solution plug is moved, the interaction with the tube is lost, and thus the movement of the plug can be smoothly performed. In addition, by preventing the electrification of the nucleic acid extraction device 1000, the nucleic acid extraction using the nucleic acid extraction device 1000 is easily automated. In this specification, the “prevention of electrification” is used as long as generated electrification is reduced to the extent that the tubes function with no problems without the need to completely eliminate the electrification.

### 1.2. First Plug, Third Plug, and Fifth Plug

The first plug 10, the third plug 30, and the fifth plug 50 are all formed of an oil. The oils of the first plug 10, the third plug 30, and the fifth plug 50 may be different kinds of oils. The liquids which form the first plug 10, the second plug 20, the third plug 30, the fourth plug 40, and the fifth plug 50 next to each other are selected so as not to mix with each other.

For example, a silicone oil or a mineral oil can be used as the oil. Here, the silicone means an oligomer or a polymer having a siloxane bond as a main skeleton. In this specification, among silicones, a silicone having a liquid form in a temperature zone which is used in the thermal cycle treatment is particularly referred to as a silicone oil. In this specification, a material which is refined from petroleum and is a liquid in a temperature zone which is used in the thermal cycle treatment is referred to as a mineral oil. Since these oils have high heat stability and products having a viscosity of 5×10³ Nsm⁻² or lower are easily available, the above mentioned oils are suitable.

Examples of the silicone oil include dimethylsilicone oils such as KF-96L-0.65cs, KF-96L-1cs, KF-96L-2cs, and KF-96L-5cs, all manufactured by Shin-Etsu Chemical Co., Ltd., SH200 C FLUID 5 CS, manufactured by Dow Corning Toray Co., Ltd., and TSF451-5A and TSF451-10, all manufactured by Momentive Performance Materials Japan LLC. Examples of the mineral oil include materials containing, as a main component, an alkane having approximately 14 to 20 carbon atoms. That is, n-tetradecane, n-pentadecane, n-hexadecane, n-heptadecane, n-octadecane, n-nonadecane, and n-tetracosane are exemplified.

As described above, an antistatic agent is preferably added to the oil. As the antistatic agent, for example, a modified silicone oil can be used. Here, the modified silicone oil means a silicone oil having a substituent. A second liquid preferably has, for example, a carbinol group, an alkylsilyl group, a fluoroalkyl group, a silanol group, or an alkylsilsesquioxy group as a substituent. The second liquid may have more than one of these substituents, or may have, for example, an alkylsilyl group and an alkylsilsesquioxy group, or an alkylsilyl group and a fluoroalkyl group. Cyclic siloxane may also be used. The second liquid preferably has heat stability in a temperature range of the thermal cycle treatment. Examples thereof include a carbinol-modified silicone oil, KF-6001 manufactured by Shin-Etsu Chemical Co., Ltd., BY 16-201 and 5562 CALBINOL FLUID all manufactured by Dow Corning Toray Co., Ltd., and XF42-B0970 manufactured by Momentive Performance Materials Japan LLC. The carbinol-modified silicone oil has a viscosity of 3×10⁴ Nsm⁻² or higher. This viscosity is too high for the carbinol-modified silicone oil to be used alone for nucleic acid extraction device. However, since the carbinol-modified silicone oil has a lower volume resistivity than a dimethylsilicone oil, the conductivity of the oil to be used can be adjusted by mixing the carbinol-modified silicone oil with a dimethylsilicone oil. That is, the larger the amount to be added, the lower the volume specific resistivity, and although the amount to be added is not particularly limited, the amount to be added is preferably adjusted so that the oil after mixing has a volume specific resistivity of 5.4×10¹⁰ Ω·cm or lower.

The antistatic agent may be a liquid containing a plurality of components, or a mixture of a plurality kinds of liquids. For example, X21-5250 (50% trimethylsiloxysilicic acid, 50% cyclopentasiloxane) and X21-5616 (60% trimethylsiloxysilicic acid, 40% isododecane), all manufactured by Shin-Etsu Chemical Co., Ltd., may be used.

The second plug 20 is disposed between the first plug 10 and the third plug 30. Another liquid plug may be disposed in an area on the side of the first plug 10 opposite the second plug 20. It is preferable that bubbles and other liquids do not exist in the first plug 10. However, as long as particles adsorbing a nucleic acid can pass through the first plug 10, bubbles and other liquids may exist. In addition, it is preferable that bubbles and other liquids do not exist between the first plug 10 and the second plug 20. However, as long as particles adsorbing a nucleic acid can pass through from the first plug 10 to the second plug 20, bubbles and other liquids may exist. Similarly, it is preferable that bubbles and other liquids do not exist between the second plug 20 and the third plug 30. However, as long as particles adsorbing a nucleic acid can pass through from the second plug 20 to the third plug 30, bubbles and other liquids may exist.

The fourth plug 40 is disposed between the third plug 30 and the fifth plug 50. Another liquid plug may be disposed in an area on the side of the fifth plug 50 opposite the fourth plug 40. It is preferable that bubbles and other liquids do not exist in the third plug 30. However, as long as particles adsorbing a nucleic acid can pass through the third plug 30, bubbles and other liquids may exist. In addition, it is preferable that bubbles and other liquids do not exist between the third plug 30 and the fourth plug 40. However, as long as particles adsorbing a nucleic acid can pass through from the third plug 30 to the fourth plug 40, bubbles and other liquids may exist. Similarly, it is preferable that bubbles and other liquids do not exist between the fourth plug 40 and the fifth plug 50. However, as long as particles adsorbing a nucleic acid can pass through from the fourth plug 40 to the fifth plug 50, bubbles and other liquids may exist. Furthermore, it is preferable that bubbles and other liquids do not exist in the fifth plug 50.

The lengths of the first plug 10, the third plug 30, and the fifth plug 50 in the longitudinal direction of the tube portion 100 are not particularly limited within a range in which the plugs can be formed. The lengths of the first plug 10, the third plug 30, and the fifth plug 50 in the longitudinal direction of the tube portion 100 are specifically 1 mm to 50 mm. In order to keep a moving distance of particles from increasing excessively, the lengths are preferably 1 mm to 30 mm, and more preferably 5 mm to 20 mm. Among these, when the length of the third plug 30 in the longitudinal direction of the tube portion 100 is increased, it is possible to make it harder for the second plug 20 to be discharged when an aspect in which the fourth plug 40 is discharged from an end on the side of the fifth plug 50 of the tube portion 100 is employed. In this case, a specific length of the third plug 30 can be set to 10 mm to 50 mm.

The first plug 10 and the fifth plug 50 function to prevent the first washing liquid (second plug 20) and the eluate (fourth plug 40) from being subjected to substance exchange with the outside air such as vaporization or from being contaminated from outside even when at least one end of the tube portion 100 is open. Therefore, even when at least one end of the tube portion 100 is open to the outside air, the volumes of the first washing liquid and the eluate can be kept constant, and thus a fluctuation in concentration of each liquid and contamination can be suppressed. Accordingly, it is possible to increase the accuracy of the concentrations of the nucleic acid and various agents in the nucleic acid extraction.

The third plug 30 functions to suppress the mixing of the first washing liquid (second plug 20) and the eluate (fourth plug 40). In addition, when the third plug 30 is formed of an oil having a higher viscosity, a “wiping effect” of the oil can be increased when particles are moved in an interface with the first washing liquid (second plug 20). Accordingly, when particles are moved from the first washing liquid plug which is the second plug 20 to the third oil plug 30, it is possible to make it harder for a water-soluble component adhering to the particles to be brought into the third plug 30 (oil).

### 1.3. Second Plug

The second plug 20 is disposed at a position between the first plug 10 and the third plug 30 in the tube portion 100. The second plug 20 is formed of a first washing liquid. The first washing liquid is a liquid which does not mix with both the oil of the first plug 10 and the oil of the third plug 30. Examples of the first washing liquid include water and a buffer solution having a solute concentration of 10 mM or lower, preferably 7 mM or lower, and more preferably 5 mM or lower. The composition of the buffer solution is not particularly limited, but a tris-hydrochloric acid buffer solution and the like can be exemplified. An ethylenediaminetetraacetic acid (EDTA) and the like may be contained. With such a first washing liquid, particles adsorbing a nucleic acid can be efficiently washed.

The volume of the second plug 20 is not particularly limited, and can be appropriately set with an amount of particles adsorbing a nucleic acid as an index. For example, when the volume of the particles is 0.5 μL, it is sufficient that the volume of the second plug 20 is 10 μL or greater, and it is preferably 20 μL to 50 μL, and more preferably 20 μL to 30 μL. When the volume of the second plug 20 is within this range, washing of the particles can be sufficiently performed when the volume of the particles is 0.5 μL. The greater the volume of the second plug 20, the more preferable to wash the particles. However, the volume of the second plug 20 can be appropriately set in consideration of the length and thickness of the tube portion 100, the length of the second plug 20 in the longitudinal direction of the tube portion 100 depending thereon, and the like.

The second plug 20 may be partitioned by oil plugs so as to be formed of a plurality of plugs. When the second plug 20 is formed of a plurality of plugs partitioned by oil plugs, a plurality of first washing liquid plugs is formed. Accordingly, it is preferable that the second plug 20 be partitioned by oil plugs since when a washing target is a water-soluble substance, a concentration of the water-soluble substance reached by a partitioned first washing liquid is lower than a concentration of the water-soluble substance reached by an unpartitioned first washing liquid having the same volume. The number of plugs into which the second plug 20 is to be partitioned is arbitrarily set. When a washing target is a water-soluble substance, and for example, the second plug 20 is partitioned into two plugs having equal volumes, a calculated concentration of the water-soluble substance can be reduced up to ¼ of a concentration of a case in which the second plug 20 is not partitioned. The number of plugs into which the second plug 20 is to be partitioned can be appropriately set in consideration of, for example, the length of the tube portion 100, the washing target, and the like.

### 1.4. Fourth Plug

The fourth plug 40 is disposed at a position between the third plug 30 and the fifth plug 50 in the tube portion 100. The fourth plug 40 is formed of an eluate.

The eluate refers to a liquid which eliminates a nucleic acid adsorbed to particles from the particles and elutes it in a liquid. Examples of the eluate include purified water such as sterilized water, distilled water, and ion-exchanged water and aqueous solutions in which at least one of an enzyme, dNTP, a probe, a primer, and a buffer is dissolved in such water. The eluate is a liquid which does not mix with both the oil of the third plug 30 and the oil of the fifth plug 50.

When the eluate is water or an aqueous solution, the nucleic acid adsorbed to particles can be isolated (eluted) by immersing the particles adsorbing the nucleic acid in the eluate. When an aqueous solution in which at least one of an enzyme, dNTP, a probe, a primer, and a buffer is dissolved is selected as the eluate, the nucleic acid adsorbed to particles can be isolated (eluted), and some or all of components necessary for a reaction liquid of PCR can be contained in the eluate. Accordingly, the time and effort for a case in which the reaction liquid of PCR is prepared using the eluate can be further saved. The concentration when at least one of an enzyme, dNTP, a probe, a primer, and a buffer is dissolved in the eluate of the fourth plug 40 is not particularly limited and can be set according to the reaction liquid of PCR to be prepared.

Here, the dNTP represents four kinds of deoxyribonucleotide triphosphates (a mixture of deoxyadenosine triphosphate (dATP), deoxycytidine triphosphate (dCTP), deoxyguanosine triphosphate (dGTP), and thymidine triphosphate (dTTP)).

The volume of the fourth plug 40 is not particularly limited, and can be appropriately set with an amount of particles adsorbing a nucleic acid as an index. For example, when the volume of the particles is 0.5 μL, it is sufficient that the volume of the fourth plug 40 is 0.5 μL or greater, and it is preferably 0.8 μL to 5 μL, and more preferably 1 μL to 3 μL. When the volume of the fourth plug 40 is within this range, elution of the nucleic acid from the particles can be sufficiently performed when the volume of the particles is 0.5 μL. To elute the nucleic acid from the particles, the volume of the fourth plug 40 can be appropriately set in consideration of the length and thickness of the tube portion 100 and rapidity of a thermal cycle of PCR so that a thermal capacity of the reaction liquid is kept from increasing excessively.

### 1.5. Advantages

The nucleic acid extraction device 1000 of this embodiment has the tube portion 100 in which the oils, the first washing liquid, and the eluate are arranged in the form of plugs. Therefore, by introducing particles adsorbing a nucleic acid to the tube portion 100 from the side of the first plug 10 and by then moving the particles up to the fourth plug 40, the nucleic acid can be easily extracted in a very short time. Specifically, by: introducing the particles adsorbing the nucleic acid from the side of the first plug 10 of the tube portion 100; passing the particles through the oil of the first plug 10; washing the particles with the first washing liquid of the second plug 20; and passing the particles through the oil of the third plug 30, the nucleic acid can be eliminated from the particles in the eluate of the fourth plug 40. That is, the nucleic acid extraction device 1000 of this embodiment can obtain an eluate containing the nucleic acid of high purity by moving the particles adsorbing the nucleic acid in the tube portion 100. Therefore, according to the nucleic acid extraction device 1000, the time and effort required for preprocessing for PCR can be significantly reduced.

### 1.6. Configuration of Nucleic Acid Extraction Device, Etc.

The nucleic acid extraction device of this embodiment has the tube portion 100, the first plug 10, the second plug 20, the third plug 30, the fourth plug 40, and the fifth plug 50. However, it may also include a configuration with other functions added thereto. The nucleic acid extraction device of this embodiment may include a combination of configurations to be described below, and modifications of the configurations.

### 1.6.1. End Portion of Tube Portion

FIG. 2 is a diagram schematically illustrating a nucleic acid extraction device 1010 which is a modification example of the nucleic acid extraction device. The nucleic acid extraction device of this embodiment may have, for example, an open end on the side of a fifth plug 50 of a tube portion 100. That is, as illustrated in FIG. 2, in the nucleic acid extraction device 1010, the end on the side of the fifth plug 50 of the tube portion 100 is open. According to the nucleic acid extraction device 1010, the fifth plug 50 and a fourth plug 40 can be discharged in order by applying a pressure to the inside of the tube portion 100 from a side of a first plug 10 of the tube portion 100. Accordingly, an eluate (fourth plug 40) containing a target nucleic acid can be easily dispensed to, for example, a reaction container for PCR using the nucleic acid extraction device 1010.

### 1.6.2. Cock

FIG. 3 is a diagram schematically illustrating a nucleic acid extraction device 1020 which is a modification example of the nucleic acid extraction device. The nucleic acid extraction device of this embodiment may further have, for example, a detachable cock 110 to seal an end on the side of a fifth plug 50 of a tube portion 100 as illustrated in FIG. 3. The cock 110 can be made of, for example, a rubber, an elastomer, or a polymer. When the tube portion 100 is sealed by the cock 110, the cock 110 may be in contact with the fifth plug 50, or a gas such as air may be disposed between the fifth plug 50 and the cock 110. Although the cock 110 is detachable, the mechanism thereof is not particularly limited. The example of FIG. 3 illustrates an aspect in which a part of the cock 110 is inserted into the tube portion 100 and fixed, but the cock 110 may have a cap form.

When the cock 110 is removed in the nucleic acid extraction device 1020, the end on the side of the fifth plug 50 of the tube portion 100 is opened, and thus the aspect of the nucleic acid extraction device 1010 of FIG. 2 is provided, and an eluate (fourth plug 40) containing a target nucleic acid can be easily dispensed to, for example, a reaction container for PCR using the nucleic acid extraction device 1020. When the cock 110 seals the end on the side of the fifth plug 50 of the tube portion 100 (FIG. 3), an effect of suppressing the movement of each plug in the tube portion 100 is obtained. Thus, for example, when particles are moved in the tube portion 100, the movement of the plug with the movement of the particles can be suppressed.

### 1.6.3. Container

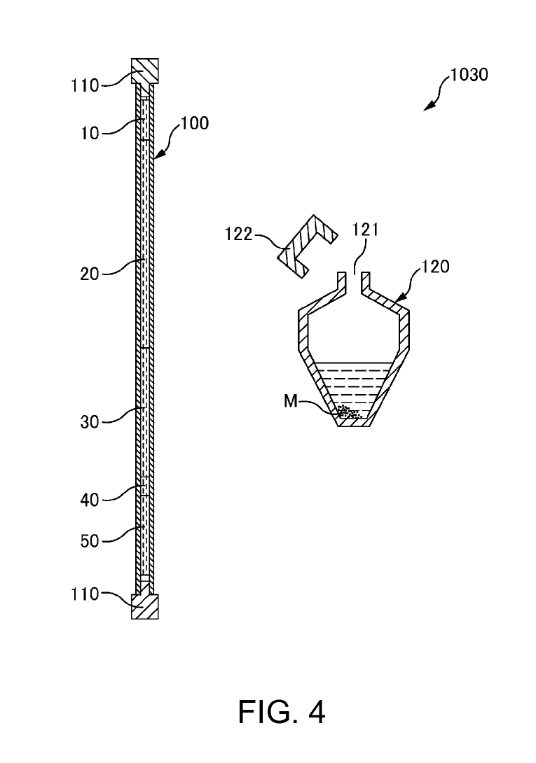

FIG. 4 is a diagram schematically illustrating a nucleic acid extraction device 1030 which is an example of the configuration of the nucleic acid extraction device. As illustrated in FIG. 4, the nucleic acid extraction device 1030 further has a detachable container 120 which can be connected to an end on the side of a first plug 10 of a tube portion 100 by internal communication.

The container 120 can be formed as a separate member. The container 120 can accommodate a liquid therein. The container 120 has an opening 121 through which a liquid and a solid can be put in and out. The example of FIG. 4 illustrates an aspect in which the opening 121 of the container 120 is connected to the end on the side of the first plug 10 of the tube portion 100 by internal communication. The container 120 may have a plurality of openings 121. In this case, an aspect in which one opening 121 is connected to the end on the side of the first plug 10 of the tube portion 100 by internal communication may be employed.

The internal volume of the container 120 is not particularly limited. However, it can be set to 0.1 mL to 100 mL. If necessary, the opening 121 of the container 120 may have such a structure as to be sealed by a lid 122. The material of the container 120 is not particularly limited, and a polymer, a metal, and the like can be used.

The opening 121 of the container 120 can be connected to the end on the side of the first plug 10 of the tube portion 100. However, the connection between the container 120 and the tube portion 100 is not particularly limited as long as the contents do not leak therefrom. When the container 120 and the tube portion 100 are connected to each other, the inside of the container 120 and the inside of the tube portion 100 can be allowed to communicate with each other. If necessary, the container 120 can be detached from the tube portion 100.

As in the nucleic acid extraction device 1030, by providing the container 120, for example, particles, an adsorbent liquid, and a sample can be accommodated in the container 120 and a nucleic acid can be adsorbed to the particles. Thereafter, when the container 120 is connected to the end on the side of the first plug 10 of the tube portion 100, the particles can be easily introduced from the side of the first plug 10 of the tube portion 100 into the tube portion 100.

The adsorbent liquid refers to a liquid which becomes a site for adsorbing a nucleic acid to particles (magnetic particles M), and is, for example, an aqueous solution containing a chaotropic agent. The adsorbent liquid may contain a chelating agent, a surfactant, and the like. Specifically, in the adsorbent liquid, disodium dihydrogen ethylenediaminetetraacetate or its dihydrate may be dissolved, or polyoxyethylene sorbitan monolaurate and the like may be contained.

Here, the chaotropic agent refers to a substance which reduces an interaction between water molecules to make the structure of the water molecules unstable. Specific examples thereof include guanidinium ions, urea, and iodide ions. When the chaotropic agent exists in the water, the nucleic acid in the water has a thermodynamical advantage when existing by being adsorbed to a solid, rather than existing by being surrounded by water molecules, and thus the nucleic acid is adsorbed to surfaces of the particles. As a substance for generating the chaotropic agent in the water, guanidinium hydrochloride, sodium iodide, and the like are exemplified.

The container 120 can be shaken in a state of being disconnected from the tube portion 100 to sufficiently stir the liquid in the container 120. Accordingly, the nucleic acid can be rapidly adsorbed to the particles. The container 120 may have the lid 122 to seal the opening 121. Furthermore, by appropriately changing the amount of the sample to be introduced to the container 120 and the volume of the liquid (particularly, fourth plug 40) in the tube portion 100, the nucleic acid in the sample can be quantitatively concentrated in the eluate of the fourth plug 40.

When a flexible material such as a rubber, an elastomer, or a polymer is selected as the material of the container 120, the inside of the tube portion 100 can be pressurized by deforming the container 120 in a state in which the container 120 is connected to the tube portion 100. Thus, when the eluate of the fourth plug 40 is discharged from an end on the side of a fifth plug 50 of the tube portion 100, the pressure is easily applied from the side of the first plug 10 of the tube portion 100. Accordingly, the eluate can be dispensed to, for example, a reaction container for PCR.

### 1.6.4. Liquid Reservoir

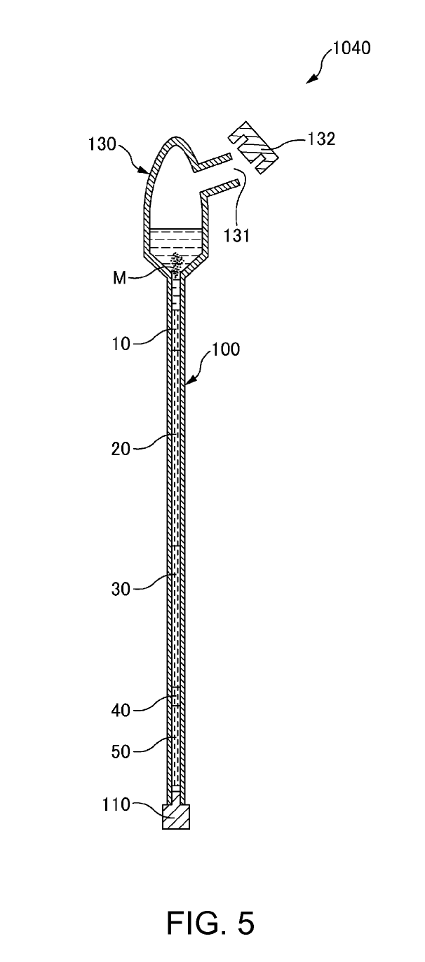

FIG. 5 is a diagram schematically illustrating a nucleic acid extraction device 1040 which is an example of the configuration of the nucleic acid extraction device. As illustrated in FIG. 5, the nucleic acid extraction device 1040 has a liquid reservoir 130 which is formed at an end on the side of a first plug 10 of a tube portion 100 to communicate with the tube portion 100. The inside of the liquid reservoir 130 and the inside of the tube portion 100 communicate with each other.

The liquid reservoir 130 can accommodate a liquid therein. The liquid reservoir 130 has an opening 131 through which a substance can be introduced from the outside to the inside of the liquid reservoir 130. The position at which the opening 131 is formed in the liquid reservoir 130 is not particularly limited. The liquid reservoir 130 may have a plurality of openings 131. The internal volume of the liquid reservoir 130 is not particularly limited. However, it can be set to 0.1 mL to 100 mL. The material of the liquid reservoir 130 is not particularly limited, and a polymer, a metal, or the like can be used. The material of the liquid reservoir 130 may be the same as that of the tube portion 100.

As in the nucleic acid extraction device 1040, by providing the liquid reservoir 130, for example, particles, an adsorbent liquid, and a sample can be accommodated in the liquid reservoir 130 and a nucleic acid can be adsorbed to the particles. The particles can be easily introduced into the tube portion 100 from the side of the first plug 10 of the tube portion 100.

In addition, the liquid reservoir 130 can be shaken together with the tube portion 100 to sufficiently stir the liquid in the liquid reservoir 130. Accordingly, the nucleic acid can be rapidly adsorbed to the particles. Furthermore, by appropriately changing the amount of the sample to be introduced to the liquid reservoir 130 and the volume of the liquid in the tube portion 100, the nucleic acid in the sample can be quantitatively concentrated in an eluate.

When the liquid reservoir 130 is provided as in the nucleic acid extraction device 1040, a detachable lid 132 may be further provided to seal the opening 131 of the liquid reservoir 130. When a flexible material such as a rubber, an elastomer, or a polymer is selected as the material of the liquid reservoir 130, the inside of the tube portion 100 can be pressurized by deforming the liquid reservoir 130 in a state in which the lid 132 is mounted on the liquid reservoir 130.

Thus, when the eluate of a fourth plug 40 in which the nucleic acid is eluted is discharged from an end on the side of a fifth plug 50 of the tube portion 100, the pressure can be easily applied from the side of the first plug 10 of the tube portion 100. Accordingly, it is possible to perform the processing ranging from a process of introducing a sample to the container 120 to a process of easily dispensing an eluate to, for example, a reaction container for PCR. In addition, when mounting the lid 132, it is possible to suppress liquid leakage when the liquid reservoir 130 is shaken together with the tube portion 100. Thus, the efficiency of adsorbing the nucleic acid to particles can be improved.

### 1.6.5. Sixth Plug and Seventh Plug

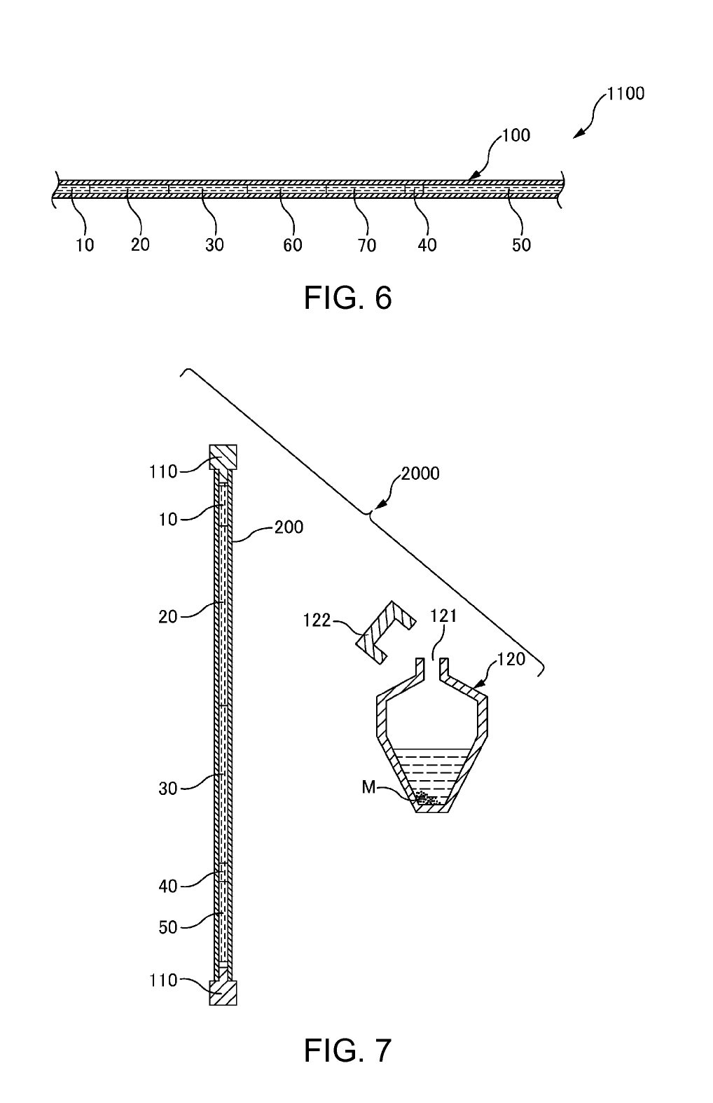

The nucleic acid extraction device of this embodiment may have a sixth plug and a seventh plug in the tube portion. FIG. 6 is a diagram schematically illustrating a nucleic acid extraction device 1100 having a sixth plug 60 and a seventh plug 70 in a tube portion 100.

The nucleic acid extraction device 1100 has a configuration in which between a third plug 30 and a fourth plug 40 in the tube portion 100 of the above-described nucleic acid extraction device, the sixth plug 60 formed of a second washing liquid which does not mix with an oil and the seventh plug 70 formed of an oil are added in order from the side of the third plug 30.

The sixth plug 60 is disposed at a position on the side of the third plug 30 in the tube portion 100 opposite a second plug 20. The sixth plug 60 is formed of a second washing liquid. The second washing liquid is a liquid which does not mix with both the oil of the third plug 30 and the oil of the seventh plug 70. Examples of the second washing liquid include water and a buffer solution having a solute concentration of 10 mM or lower, preferably 7 mM or lower, and more preferably 5 mM or lower. The composition of the buffer solution is not particularly limited, but a tris-hydrochloric acid buffer solution and the like can be exemplified. An ethylenediaminetetraacetic acid (EDTA) and the like may be contained. The second washing liquid may have a composition which is the same as or different from that of the first washing liquid.

The volume of the sixth plug 60 is not particularly limited, and can be appropriately set with an amount of particles adsorbing a nucleic acid as an index. For example, when the volume of the particles is 0.5 μL, it is sufficient that the volume of the sixth plug 60 is 10 μL or greater, and it is preferably 20 μL to 50 μL, and more preferably 20 μL to 30 μL. When the volume of the sixth plug 60 is within this range, washing of the particles can be sufficiently performed when the volume of the particles is 0.5 μL. The greater the volume of the sixth plug 60, the more preferable to wash the particles. However, the volume of the sixth plug 60 can be appropriately set in consideration of the length and thickness of the tube portion 100, the length of the sixth plug 60 in the longitudinal direction of the tube portion 100 depending thereon, and the like.

The sixth plug 60 may be partitioned by oil plugs so as to be formed of a plurality of plugs. When the sixth plug 60 is formed of a plurality of plugs partitioned by oil plugs, a plurality of second washing liquid plugs is formed. Accordingly, it is preferable that the sixth plug 60 be partitioned by oil plugs since when a washing target is a water-soluble substance, a concentration of the water-soluble substance reached by a partitioned second washing liquid is lower than a concentration of the water-soluble substance reached by an unpartitioned second washing liquid having the same volume. The number of plugs into which the sixth plug 60 is to be partitioned is arbitrarily set. When a washing target is a water-soluble substance, and for example, the sixth plug 60 is partitioned into two plugs having equal volumes, a calculated concentration of the water-soluble substance can be reduced up to ¼ of a concentration of a case in which the sixth plug 60 is not partitioned. The number of plugs into which the sixth plug 60 is to be partitioned can be appropriately set in consideration of, for example, the length of the tube portion 100, the washing target, and the like. When the first washing liquid of the second plug 20 is the same as the second washing liquid of the sixth plug 60, similar effects to those of a case in which the second plug 20 is partitioned in a nucleic acid extraction device which does not have the above-described sixth plug 60 and seventh plug 70 are obtained.

The seventh plug 70 is formed of an oil which does not mix with the liquids of the sixth plug 60 and the fourth plug 40 next thereto. The oil of the seventh plug 70 may be a different kind of oil from the oils of the first plug 10, the third plug 30, and the fifth plug 50. Examples of the oil include those exemplified in the case of the first plug 10 and the like.

It is preferable that bubbles and other liquids do not exist in the seventh plug 70. However, as long as particles adsorbing a nucleic acid can pass through the seventh plug 70, bubbles and other liquids may exist. In addition, it is preferable that bubbles and other liquids do not exist between the fourth plug 40 and the sixth plug 60 next to the seventh plug 70. However, as long as particles adsorbing a nucleic acid can be moved in the tube portion 100, bubbles and other liquids may exist. It is preferable that bubbles and other liquids do not exist in the seventh plug 70.

The length of the seventh plug 70 in the longitudinal direction of the tube portion 100 is not particularly limited within a range in which the plug can be formed. The length of the seventh plug 70 in the longitudinal direction of the tube portion 100 is specifically 1 mm to 50 mm. In order to keep a moving distance of particles from increasing excessively, the length is preferably 1 mm to 30 mm, and more preferably 5 mm to 20 mm. In the nucleic acid extraction device 1100, when the length of the seventh plug 70 in the longitudinal direction of the tube portion 100 is increased, it is possible to make it harder for the sixth plug 60 to be discharged when an aspect in which the fourth plug 40 is discharged from an end on the side of the fifth plug 50 of the tube portion 100 is employed. In this case, a specific length of the seventh plug 70 can be set to 10 mm to 50 mm.

In addition, the seventh plug 70 functions to suppress the mixing of the second washing liquid (sixth plug 60) and the eluate (fourth plug 40). In addition, when the seventh plug 70 is formed of an oil having a higher viscosity, a “wiping effect” of the oil can be increased when particles are moved in an interface with the second washing liquid (sixth plug 60). Accordingly, when particles are moved from the second washing liquid plug which is the sixth plug 60 to the seventh oil plug 70, it is possible to make it harder for a water-soluble component adhering to the particles to be brought into the seventh plug 70 (oil).

According to the nucleic acid extraction device 1100, particles adsorbing a nucleic acid can be washed in the second plug 20 and the sixth plug 60. Therefore, efficiency of washing the particles can be further increased.

In the nucleic acid extraction device 1100, the first washing liquid of the second plug 20 may contain a chaotropic agent. For example, when the first washing liquid of the second plug 20 contains guanidinium hydrochloride, it is possible to wash the particles while maintaining or strengthening the adsorption of the nucleic acid adsorbed to the particles in the second plug 20. The concentration when the second plug 20 contains guanidinium hydrochloride can be set to, for example, 3 mol/L to 10 mol/L, and preferably 5 mol/L to 8 mol/L. When the concentration of the guanidinium hydrochloride is within this range, it is possible to wash other foreign substances while more stably adsorbing the nucleic acid adsorbed to the particles.

When the second washing liquid of the sixth plug 60 is water or a buffer solution, it is possible to more stably adsorb the nucleic acid adsorbed to the particles and also possible to perform washing in the second plug 20 (first washing liquid), and it is possible to further wash the particles while diluting the chaotropic agent in the sixth plug 60 (second washing liquid).

It should be easily understood that the nucleic acid extraction device 1100 having the sixth plug 60 and the seventh plug 70 in the tube portion 100 may also have a configuration with the above-described cock, container, liquid reservoir, and the like added thereto, and thus similar effects to those described above are obtained.

### 2. NUCLEIC ACID EXTRACTION KIT

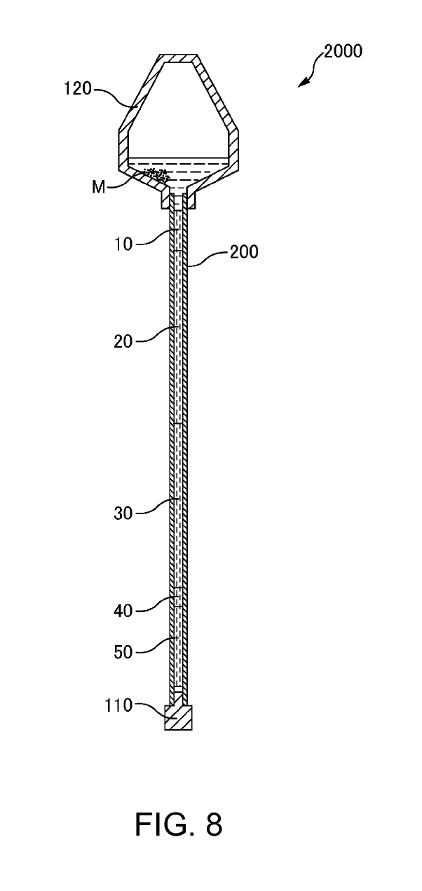

FIG. 7 is a schematic diagram illustrating an example of a nucleic acid extraction kit of this embodiment. A nucleic acid extraction kit 2000 illustrated in FIG. 7 includes components constituting the main part of the above-described nucleic acid extraction device. Similar configurations to those described in the clause “1. Nucleic Acid Extraction Device” will be denoted by the same reference numerals, and detailed description thereof will be omitted.

The nucleic acid extraction kit 2000 of this embodiment includes a tube 200 in which a first plug 10 formed of an oil, a second plug 20 formed of a first washing liquid which does not mix with an oil, a third plug 30 formed of an oil, a fourth plug 40 formed of an eluate which does not mix with an oil, and a fifth plug 50 formed of an oil are arranged in this order, and a container 120 which can be connected to an end on the side of the first plug 10 of the tube 200 by internal communication.

The tube 200 has an aspect in which both ends of the tube portion 100 of the nucleic acid extraction device 1000 are open. The tube 200 has a cavity therein and has a tubular shape in which a liquid can be allowed to flow in a longitudinal direction in the cavity. The internal shape, external shape, size, properties, material, and the like of the tube 200 are similar to those of the tube portion 100 of the nucleic acid extraction device 1000. The plugs arranged in the tube 200 are similar to the plugs arranged in the tube portion 100 of the nucleic acid extraction device 1000. Both ends of the tube 200 may be sealed by detachable cocks 110. When both the ends of the tube 200 are sealed by the cocks 110, for example, storage and transfer of the nucleic acid extraction kit 2000 are facilitated. Furthermore, when the cock 110 seals an end on the side of the fifth plug 50 of the tube 200 during the use of the tube 200, the movement of each plug in the tube 200 can be suppressed when particles are moved in the tube 200, and thus washing and extraction can be further facilitated. Since the cock 110 is detachable, the end on the side of the fifth plug 50 of the tube 200 can be opened, and thus the eluate of the fourth plug 40 in which a nucleic acid is eluted is easily discharged from the end on the side of the fifth plug 50 of the tube 200.

The container 120 is similar to the container 120 described in the clause of the nucleic acid extraction device 1000.

In the example of FIG. 7, both the ends of the tube 200 are sealed by the detachable cocks 110. The nucleic acid extraction kit 2000 may include a lid 122 which detachably seals an opening 121 of the container 120, or the opening 121 of the container 120 may be sealed by the detachable lid 122. The nucleic acid extraction kit 2000 may accommodate some or all of components of an adsorbent liquid in the container 120.

In addition, in the nucleic acid extraction kit 2000, the container 120 may accommodate an adsorbent liquid and magnetic particles. Thus, a process of adsorbing, when a sample is introduced into the container 120, a nucleic acid contained in the sample to the magnetic particles can be performed in the container 120. Accordingly, it is possible to more rapidly perform preprocessing of PCR without the need to provide another container. In addition, in this case, the opening 121 of the container 120 may be sealed by the detachable lid 122 if necessary. The magnetic particles will be described later in detail.

When the container 120 is made of a flexible material as described above, the inside of the tube 200 can be pressurized by deforming the container 120 in a state in which the container 120 is connected to the tube 200. Thus, when the eluate of the fourth plug 40 in which the nucleic acid is eluted is discharged from the end on the side of the fifth plug 50 of the tube 200, the pressure can be easily applied from the side of the first plug 10 of the tube 200. Accordingly, it is possible to easily dispense the eluate to, for example, a reaction container for PCR.

The nucleic acid extraction kit 2000 may include other configurations such as a cock, a lid, an instruction manual, a reagent, a case, other than the tube 200 and the container 120. Here, an example has been shown in which five plugs are arranged in the tube 200. However, as described in the clause “1.6. Nucleic Acid Extraction Device”, it should be easily understood that if necessary, other plugs such as the sixth plug 60 and the seventh plug 70 may be arranged in the tube 200 (tube portion 100).

The nucleic acid extraction kit 2000 of this embodiment has the container 120 which can be connected to the end on the side of the first plug 10 of the tube 200 by internal communication. Accordingly, when particles and a sample are accommodated in the container 120, the nucleic acid can be adsorbed to the particles, and thus the particles can be easily introduced into the tube 200 from the side of the first plug of the tube 200 when the container 120 is connected to the end on the side of the first plug 10 of the tube 200. In addition, since the nucleic acid extraction kit 2000 of this embodiment has the container 120, the container 120 can be shaken, and thus the liquid in the container 120 can be sufficiently stirred. Accordingly, the nucleic acid can be rapidly adsorbed to the particles.

When connecting the container 120 to the tube 200, the particles adsorbing the nucleic acid are easily moved up to the fourth plug 40 by introducing the particles from the end on the side of the first plug 10 of the tube 200. Accordingly, the nucleic acid can be easily extracted in a very short time. The nucleic acid extraction kit 2000 can obtain an eluate containing the nucleic acid of high purity by moving the particles adsorbing the nucleic acid into the tube 200. Therefore, according to the nucleic acid extraction kit 2000, the time and effort required for preprocessing for PCR can be significantly reduced.

### 3. NUCLEIC ACID EXTRACTION METHOD

All of the nucleic acid extraction device, the nucleic acid extraction kit, and the modifications thereof, which have been described above, and a nucleic acid extraction apparatus to be described later can be appropriately used in a nucleic acid extraction method of this embodiment. Hereinafter, a method using the above-described nucleic acid extraction kit 2000 will be described as an example of the nucleic acid extraction method of this embodiment.

The nucleic acid extraction method of this embodiment includes: a process of introducing a sample containing a nucleic acid to the flexible container 120 accommodating magnetic particles M and an adsorbent liquid; a process of adsorbing the nucleic acid to the magnetic particles M by vibrating the container 120; a process of connecting the container 120 to the end on the side of the first plug 10 of the tube 200, in which the first plug 10 formed of an oil, the second plug 20 formed of a first washing liquid which does not mix with an oil, the third plug 30 formed of an oil, the fourth plug 40 formed of an eluate which does not mix with an oil, and the fifth plug 50 formed of an oil are arranged in this order, by allowing the inside of the container 120 and the inside of the tube 200 to communicate with each other; a process of passing the magnetic particles M through the tube 200 from the container 120 by applying a magnetic force to move the magnetic particles M up to the position of the fifth plug 50; and a process of eluting the nucleic acid from the magnetic particles M in the eluate of the fourth plug 40.

In the nucleic acid extraction method of this embodiment, various particles (for example, silica particles, polymer particles, magnetic particles, or the like) can be used as long as the particles can adsorb the nucleic acid using an adsorbent liquid and can be moved in the tube 200. However, in an embodiment of the nucleic acid extraction method to be described below, the magnetic particles M which are particles containing a magnetic body and can adsorb the nucleic acid to surfaces of the particles are used. When particles other than the magnetic particles M are moved in the tube, this movement can be performed using, for example, gravity or a potential difference.

In the nucleic acid extraction method of this embodiment, a material transmitting a magnetic force is selected for the container 120 and the tube 200, and a magnetic force is applied from the outside of the container 120 and the tube 200 to move the magnetic particles Min the container 120 and the tube 200.

The sample contains a nucleic acid which becomes a target. Hereinafter, it may be simply referred to as a target nucleic acid. The target nucleic acid is, for example, DNA and/or RNA (DNA: Deoxyribonucleic Acid, and/or RNA: Ribonucleic Acid). The target nucleic acid is extracted from the sample through the nucleic acid extraction method of this embodiment, is eluted in the eluate, and is then used as, for example, a template of PCR. Examples of the sample include blood, nasal cavity mucous, oral mucous membrane, and other various biological samples.

### 3.1 Process of Introducing Sample to Container

The process of introducing the sample to the container 120 can be performed in such a manner that for example, the sample is attached to a swab and the swab is put from the opening 121 of the container 120 to immerse the swab in the adsorbent liquid. The sample may be introduced from the opening 121 of the container 120 using a pipette or the like. When the sample has a paste form or a solid form, the sample may be attached or fed to an internal wall of the container 120 using a spoon, tweezers, or the like from the opening 121 of the container 120.

### 3.2. Process of Adsorbing Nucleic Acid to Magnetic Particles

The process of adsorbing the nucleic acid is performed by vibrating the container 120. When there is the lid 122 which seals the opening 121 of the container 120, this process can be more efficiently performed by sealing the container 120 using the lid 122. Through this process, the target nucleic acid is adsorbed to surfaces of the magnetic particles M by an action of a chaotropic agent. In this process, a nucleic acid other than the target nucleic acid or protein may be adsorbed to the surfaces of the magnetic particles M.

As the method of vibrating the container 120, an apparatus such as a vortex shaker may be used, or the container 120 may be shaken by operator's hand. A magnetic property of the magnetic particles M may be used to vibrate the container 120 while giving a magnetic field from the outside. The time for vibration of the container 120 can be appropriately set. For example, when the shape of the container 120 is roughly a cylindrical shape having a diameter of approximately 20 mm and a height of approximately 30 mm, stirring is sufficiently performed by shaking and vibrating the container 120 for 10 seconds by hand to adsorb the nucleic acid to the surfaces of the magnetic particles M.

### 3.3. Process of Connecting Container to Tube

Next, the container 120 is connected to the end on the side of the first plug 10 of the tube 200 as illustrated in FIG. 8. Each plug in the tube 200 is difficult to move in the tube 200 since the cock 110 on the side of the seventh plug 70 is not removed even when the cock 110 on the side of the first plug 10 is removed. When the cock 110 is attached to the end on the side of the first plug 10 of the tube 200, this process is performed after removing the cock 110. The container 120 and the tube 200 are connected to each other so that the contents do not leak therefrom, and are thus allowed to communicate with each other so that the contents can be allowed to flow between the inside of the container 120 and the inside of the tube 200.

### 3.4. Process of Moving Magnetic Particles

Through the above-described processes, the magnetic particles M adsorbing the nucleic acid in the container 120 enter into a state in which these can be allowed to flow in the tube 200. As the method of introducing the magnetic particles M adsorbing the nucleic acid to the tube 200, a method using gravity or a centrifugal force may be used. The introduction method is not particularly limited, but in this embodiment, the introduction is performed by applying a magnetic force from the outside of the container 120 and the tube 200. The magnetic force can be applied using, for example, a permanent magnet, an electromagnet, or the like. However, it is preferable that the magnetic force be applied using a permanent magnet because heat and the like are not generated. When a permanent magnet is used, the introduction may be performed by moving the magnet by operator's hand, or a mechanical device or the like may be used. The magnetic particles M have such a property as to be pulled by a magnetic force. Accordingly, using this property, the relative arrangement between: the container 120 and the tube 200; and the permanent magnet is changed to move the magnetic particles M from the inside of the container 120 to the tube 200. Thus, the magnetic particles M are moved from the first plug 10 to the fourth plug 40 through the plugs in order. A staying time in each plug when the magnetic particles M pass through each plug is not particularly limited, and the magnetic particles M may be moved to reciprocate in the longitudinal direction of the tube 200 in the same plug.

### 3.5. Process of Eluting Nucleic Acid

When the magnetic particles M reach the fourth plug 40, the nucleic acid adsorbed to the magnetic particles M is eluted to the eluate of the fourth plug 40 by the action of the eluate. Through this process, the nucleic acid is eluted from the sample to the eluate, and a state in which the nucleic acid is eluted from the sample is made.

### 3.6. Advantages

According to the nucleic acid extraction method of this embodiment, it is possible to easily extract a nucleic acid in a very short time. In the nucleic acid extraction method of this embodiment, magnetic particles M adsorbing a nucleic acid are moved in the tube 200, and thus it is possible to obtain an eluate containing the nucleic acid of high purity. According to the nucleic acid extraction method of this embodiment, the time and effort required for preprocessing for PCR can be significantly reduced.

### 3.7. Process of Discharging Fourth Plug from Tube

The nucleic acid extraction method of this embodiment may include a process of discharging the fifth plug 50 and the fourth plug 40 from an end of the tube 200 on the side opposite to the end connected to the container 120 by deforming the container 120.

This process can be performed by deforming the container 120 after the process described in the clause “3.5. Process of Eluting Nucleic Acid”. When the fourth plug 40 is discharged, the fifth plug 50 is discharged first. The cock 110 sealing the side of the fifth plug 50 of the tube 200 is removed prior to this process to open the end on the side of the fifth plug 50 of the tube 200.

When an external force is applied to the container 120 to increase the internal pressure and the container 120 is thus deformed, each plug is moved from the side of the first plug 10 to the side of the fifth plug 50 of the tube 200 due to the pressure. Accordingly, the fifth plug 50 and the fourth plug 40 are discharged in order from the end on the side of the fifth plug 50 of the tube 200. The third plug 30 (or the seventh plug 70) may be discharged. However, the second plug 20 (or the sixth plug 60) is not permitted to be discharged. In this case, for example, when the volume of the third plug 30 (or the seventh plug 70) is set to be larger than those of other plugs and the length of the third plug 30 (or the seventh plug 70) in the longitudinal direction of the tube 200 is increased, the second plug 20 (or the sixth plug 60) is easily prevented from being discharged.

The fourth plug 40 and the fifth plug 50 are discharged to, for example, a reaction container for PCR. Therefore, the eluate and the oil are dispensed to the reaction container for PCR. Usually, the oil has no influence on PCR, and thus for example, an oil of the same kind as the oil of the fifth plug 50 may be accommodated in advance in the reaction container of PCR. In that case, when this process is performed in a state in which the tip end of the tube 200 is in the oil, the eluate containing the target nucleic acid can be introduced to the reaction container of PCR without the contact of the eluate with the outside air. When the nucleic acid extraction method of this embodiment includes this process, the eluate containing the target nucleic acid can be easily dispensed to, for example, a reaction container for PCR.

### 3.8. Modification Examples

### 3.8.1. Modification of Process of Moving Magnetic Particles

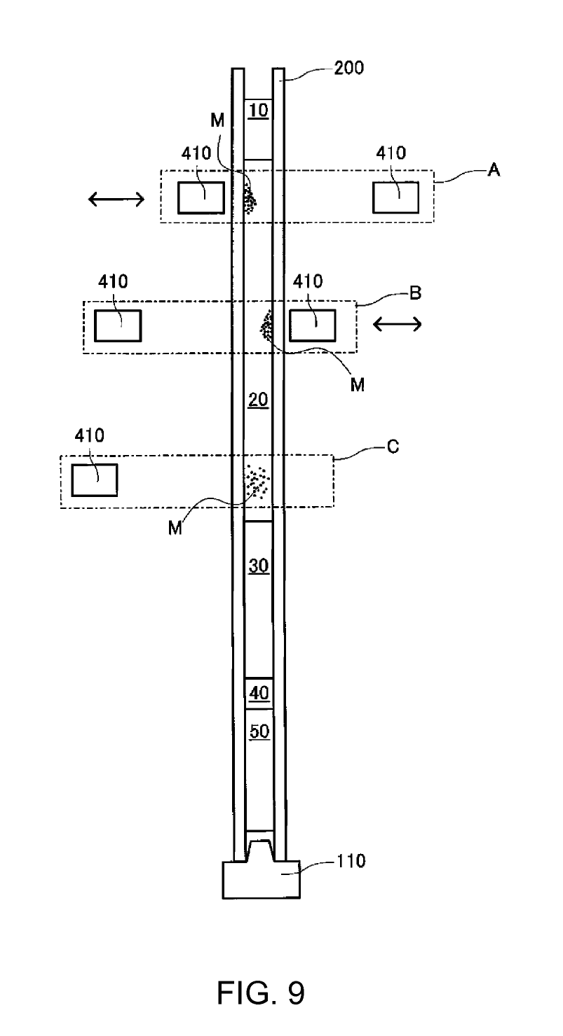

FIG. 9 is a schematic diagram for illustrating a modification of the nucleic acid extraction method of this embodiment.

In the above-described clause “3.4. Process of Moving Magnetic Particles”, the magnetic particles M pass through the plugs from the first plug 10 and are moved up to the fourth plug 40 by applying a magnetic force to the magnetic particles M from the outside. However, when the magnetic particles M are moved to the second plug 20, the magnetic particles M may be vibrated in the second plug 20, or may be repeatedly diffused and aggregated by changing the magnetic force applied from the outside. Thus, the effect of washing the magnetic particles M with the first washing liquid of the second plug 20 can be increased.

Specifically, as shown in the boxes A and B of FIG. 9, in a case in which a pair of permanent magnets 410 is used as a unit which applies a magnetic force, when the magnetic particles M are moved from the container 120, pass through the first plug 10, and reach the second plug 20 using the permanent magnets 410, the magnetic particles M can be vibrated in a direction crossing the longitudinal direction of the tube 200 in the second plug 20 when one permanent magnet 410 is moved away from the tube 200 and the other permanent magnet 410 is moved closer from the side opposed to the tube 200 (repetition of the aspects denoted by A and B of FIG. 9). Thus, the effect of washing the magnetic particles M with the first washing liquid of the second plug 20 can be increased. When the second plug 20 is partitioned or the sixth plug 60 is disposed in the tube 200, such washing of the magnetic particles M may also be applied in a plurality of second plugs 20 or in the sixth plug 60.

In addition, as shown in the box C of FIG. 9, the magnetic particles M can be diffused in the second plug 20 by simply moving the permanent magnet 410 away from the tube 200. Since the magnetic particles M have a hydrophilic surface, the magnetic particles M have difficulty entering the oils of the first plug 10 and the third plug 30 even when, for example, the magnetic force is weakened and diffused in the second plug 20. Thus, such an aspect may be employed.

Specifically, when the magnetic particles M are moved from the container 120, pass through the first plug 10, and reach the second plug 20 using the permanent magnet 410, the permanent magnet 410 is moved away from the tube 200 to diffuse the magnetic particles M in the second plug 20. The magnetic particles M can be moved again, pass through the third plug 30, and be introduced to the fourth plug 40 using the magnetic force of the permanent magnet 410.

The aspect in which the magnetic particles M are vibrated, or repeatedly diffused and aggregated by changing the magnetic force applied from the outside may also be applied when the magnetic particles M exist in the adsorbent liquid in the container 120 or when the magnetic particles M exist in the fourth plug 40 (eluate).

### 3.8.2. Modification of Process of Eluting Nucleic Acid

In the above-described clause “3.5. Process of Eluting Nucleic Acid”, the fourth plug 40 may be heated. Examples of the method of heating the fourth plug 40 include a method of bringing a heating medium such as a heating block into contact with a position corresponding to the fourth plug 40 of the tube 200, a method using a heat source such as a heater, and an electromagnetic heating method.

When the fourth plug 40 is heated, a plug other than the fourth plug 40 may be heated. However, in a state in which the magnetic particles M adsorbing the nucleic acid exist in the washing liquid plug, the plug is preferably not heated. The temperature which is reached when the fourth plug 40 is heated is preferably 35° C. to 85° C., more preferably 40° C. to 80° C., and even more preferably 45° C. to 75° C. from the viewpoint of elution efficiency and from the viewpoint of suppression of, when the eluate contains an enzyme for PCR, deactivation of the enzyme.

In the process of eluting the nucleic acid, when the fourth plug 40 is heated, the nucleic acid adsorbed to the magnetic particles M can be more efficiently eluted to the eluate. Even when the first or second washing liquid has a composition which is the same as or similar to the composition of the eluate, the nucleic acid remaining on and adsorbed to the magnetic particles M without being eluted to the washing liquid can be eluted to the eluate. That is, even after the magnetic particles M adsorbing the nucleic acid are washed with the first or second washing liquid, the nucleic acid can be further eluted to the eluate. Accordingly, sufficient washing and elution to the eluate at a sufficient concentration can be balanced even when the composition of the washing liquid and the composition of the eluate are the same as or similar to each other.

### 3.8.3. Modification of Process of Discharging Fourth Plug from Tube

When the above-described “3.7. Process of Discharging Fourth Plug from Tube” is employed, the magnetic particles M from which the adsorbed nucleic acid has been eluted to the eluate in the related process may exist in the fourth plug 40, but the magnetic particles M may be moved to any one of the first plug 10, the second plug 20, and the third plug 30 or in the container 120 by applying a magnetic force. Thus, the fourth plug 40 can be discharged from the tube 200 in a state in which the eluate does not contain the magnetic particles M. When a place where the magnetic particles M are moved is the second plug 20 or the container 120, the magnetic particles M have difficulty entering the oil of the third plug 30 even when the magnetic force is removed, and thus the fourth plug 40 can be more easily discharged from the tube 200.

### 4. NUCLEIC ACID EXTRACTION APPARATUS

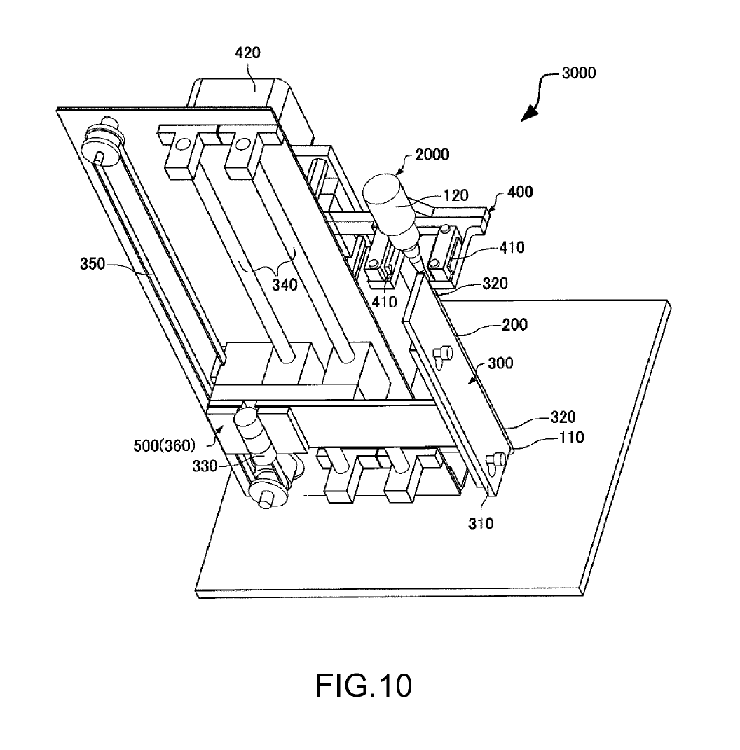

A nucleic acid extraction apparatus according to this embodiment can be appropriately applied to the nucleic acid extraction device, the nucleic acid extraction kit, and the nucleic acid extraction method which have been described above. Hereinafter, a nucleic acid extraction apparatus 3000 which extracts a nucleic acid with the nucleic acid extraction kit 2000 mounted thereon will be described as an embodiment. FIG. 10 is a perspective view schematically illustrating the nucleic acid extraction apparatus 3000 of this embodiment.

The nucleic acid extraction apparatus 3000 of this embodiment includes: a mounting portion 300 on which a tube having a longitudinal direction in which a first plug 10 formed of an oil, a second plug 20 formed of a first washing liquid which does not mix with an oil, a third plug 30 formed of an oil, a fourth plug 40 formed of an eluate which does not mix with an oil, and a fifth plug 50 formed of an oil are arranged in this order is mounted; a magnetic force application portion 400 which applies, when a tube 200 is mounted on the mounting portion 300, a magnetic force from a side surface of the tube 200; and a moving mechanism 500 which changes the relative arrangement between the mounting portion 300 and the magnetic force application portion 400 in the longitudinal direction of the tube 200.

The tube 200 mounted on the mounting portion 300 of the nucleic acid extraction apparatus 3000 is the above-described tube 200. The nucleic acid extraction apparatus 3000 has the mounting portion 300 on which the tube 200 is mounted. Although an example has been shown in which the plugs ranging from the first plug 10 to the fifth plug 50 are arranged in the tube 200, the above-described sixth plug 60 and seventh plug 70 may also be arranged.

The mounting portion 300 is a portion in which the tube 200 is mounted. Together with the tube 200, a container 120 connected to the tube 200 may also be mounted on the mounting portion 300. As the mounting portion 300, a mechanism or the like for configuration or mounting can be appropriately designed within a range in which the magnetic force application portion 400 can apply a magnetic force to the tube 200, and if necessary, to the container 120. The mounting portion 300 may be configured so that when the tube 200 is flexibly bent, the tube 200 can be mounted by being stretched into a linear shape. In addition, in the example illustrated in FIG. 10, the mounting portion 300 has a doubling plate 310 disposed along the tube 200. The doubling plate 310 is not an essential configuration. However, in some cases, vibration of the tube 200 can be suppressed when the doubling plate 310 is installed. In the example illustrated in FIG. 10, the mounting portion 300 has clip mechanisms 320, and thus the tube 200 is fixed at two places.

The mounting portion 300 is configured so that the positional relation with the magnetic force application portion 400 is relatively changed in the longitudinal direction of the tube 200. Accordingly, when a design in which the mounting portion 300 is relatively moved with respect to the magnetic force application portion 400 with no movement of the magnetic force application portion 400 is provided, a moving mechanism 360 which moves the mounting portion 300 is included as the moving mechanism 500 as illustrated in FIG. 10. In some cases, the moving mechanism 360 is not required for the mounting portion 300 when the magnetic force application portion 400 includes a moving mechanism. In the example illustrated in FIG. 10, the mounting portion 300 is configured to include a hinge 330, guide rails 340, a drive belt 350, and a motor (not illustrated).

In the example of the nucleic acid extraction apparatus 3000, one mounting portion 300 is provided, but more than one mounting portion 300 may be provided. In that case, more than one magnetic force application portion 400 can be provided, and the plural mounting portions 300 may be provided separately or in conjunction with each other.

The magnetic force application portion 400 is a configuration for applying a magnetic force to the tube 200, and if necessary, to the container 120 when the tube 200 is mounted on the mounting portion 300. The magnetic force application portion 400 is configured to include, for example, a permanent magnet, an electromagnet, or a combination thereof. The magnetic force application portion 400 is provided with at least one magnet. However, more than one magnet may be provided. It is preferable that an electromagnet not be used, but a permanent magnet be used in the magnetic force application portion 400 since generation of heat and the like are difficult to occur. As the permanent magnet, for example, a nickel-based, iron-based, cobalt-based, samarium-based, or neodymium-based permanent magnet can be used.

The magnetic force application portion 400 functions to apply a magnetic force to magnetic particles M which exist in the container 120 and in the tube 200. The magnetic particles M can be moved in the container 120 and in the tube 200 by changing the relative positional relation between the mounting portion 300 and the magnetic force application portion 400.

In the example illustrated in FIG. 10, the magnetic force application portion 400 has a pair of permanent magnets 410 provided opposed to each other with the container 120 and the tube 200 interposed therebetween. The pair of permanent magnets 410 is separated from each other at an interval larger than an external diameter of the tube 200. The direction of the polarity of the permanent magnet 410 is not particularly limited. The magnetic force application portion 400 is configured so that the positional relation with the mounting portion 300 is relatively changed in the longitudinal direction of the tube 200. Accordingly, when a design in which the magnetic force application portion 400 is relatively moved with respect to the mounting portion 300 with no movement of the mounting portion 300 is provided, a moving mechanism which moves the magnetic force application portion 400 is included as the moving mechanism 500.

In addition, in the example illustrated in FIG. 10, the magnetic force application portion 400 is disposed so that when one of the pair of permanent magnets 410 is moved closer to the tube 200, the other one is separated from the tube 200. Vibration can be applied using a motor 420 so that the pair of permanent magnets 410 is moved closer to or separated from the tube 200. The magnetic particles M can be moved to reciprocate in a direction crossing the longitudinal direction of the tube 200 in the tube 200 by driving the motor 420.

If necessary, the motor 420 can also be driven when a magnetic force is applied to any of the container 120 and the tube 200. Efficiency of washing the magnetic particles M in the tube 200 or elution efficiency can be increased when the motor 420 is driven at the time when the permanent magnet 410 is positioned at the position of the second plug 20 or the position of the fourth plug 40 of the tube 200.

According to the nucleic acid extraction apparatus 3000 of this embodiment, preprocessing for PCR can be automated, and the time and effort required for the preprocessing can be significantly reduced. In addition, according to the nucleic acid extraction apparatus 3000 of this embodiment, since the magnetic force application portion 400 can be vibrated, washing (purification) of magnetic particles M adsorbing a nucleic acid can be more efficiently performed, and thus the accuracy of PCR can be further increased.

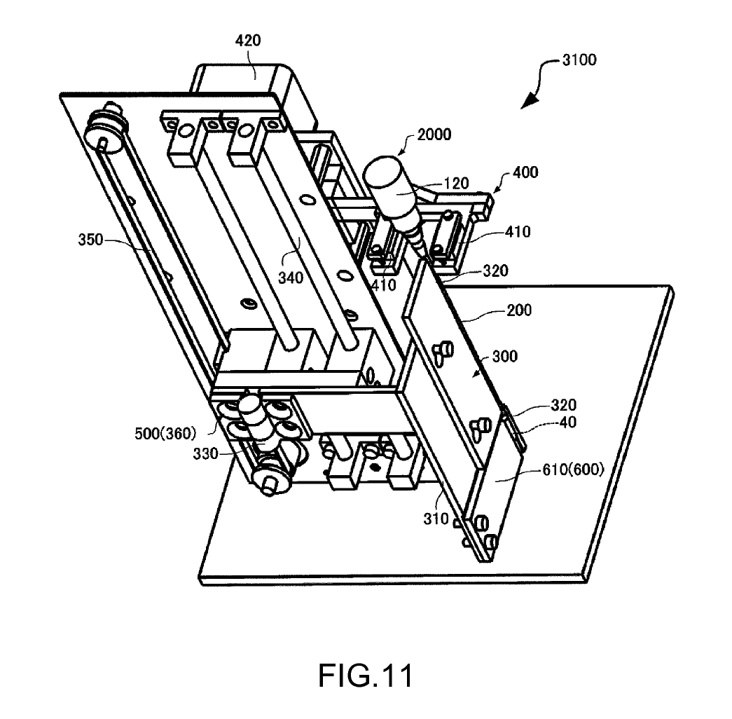

FIG. 11 is a perspective view schematically illustrating a nucleic acid extraction apparatus 3100 according to a modification example of the nucleic acid extraction apparatus. The nucleic acid extraction apparatus 3100 is the same as the above-described nucleic acid extraction apparatus 3000, except that a heating portion 600 is provided. Members having common actions and functions will be denoted by the same reference numerals, and description thereof will be omitted.

The heating portion 600 is configured to heat a part of the tube 200 when the tube 200 is mounted on the mounting portion 300. Examples of the heating portion 600 include a heat source, a heating block, a heater, and a coil for electromagnetic heating. The heating portion 600 is shaped to allow insertion of the tube 200 therein or to be brought into contact with the side surface of the tube 200, and is arbitrarily shaped as long as the liquid in the tube 200 can be heated.

The part heated by the heating portion 600 in the tube 200 includes a part where the fourth plug 40 exists in the longitudinal direction of the tube 200. The heating portion 600 may heat another part of the tube 200, but preferably does not heat apart where the second plug 20 exists in the longitudinal direction of the tube 200.

The nucleic acid extraction device 3100 illustrated in FIG. 11 is provided with, as the heating portion 600, a heater 610 which is provided in parallel to the doubling plate 310 to heat a position including the fourth plug 40 of the tube 200. The heater 610 is shaped to be brought into contact with approximately half of the outer periphery of the tube 200.

The nucleic acid extraction device 3100 can elute a sufficient amount of a nucleic acid to the eluate of the fourth plug 40 even when the amount of the nucleic acid adsorbed to magnetic particles M is reduced by washing with at least one of the first washing liquid of the second plug 20 and the second washing liquid of the sixth plug 60. Accordingly, the washing effect can be increased, and a sufficient concentration of a nucleic acid for PCR can be eluted to the eluate.

### 5. EXPERIMENTAL EXAMPLES

Hereinafter, experimental examples will be described to explain the invention in further detail. However, the invention is not limited to the following experimental examples.

### 5.1. Experimental Example 1

Experimental Example 1 used a configuration in which the plugs ranging from the first plug 10 to the seventh plug 70 are provided in the tube 200 in the above-described nucleic acid extraction kit 2000.

First, 375 μL of an adsorbent liquid and 1 μL of a magnetic bead dispersion liquid were accommodated in a polyethylene container having a capacity of 3 mL. The adsorbent liquid has a composition of 76 mass % of guanidinium hydrochloride, 1.7 mass % of disodium dihydrogen ethylenediaminetetraacetate dihydrate, and 10 mass % of an aqueous solution of polyoxyethylene sorbitan monolaurate (manufactured by Toyobo Co., Ltd., MagExtractor-Genome-, NPK-1). As the magnetic bead dispersion liquid, a liquid containing 50 vol % of magnetic silica particles and 20 mass % of lithium chloride was used.

50 μL of blood collected from a person was put from an inlet of the container using a pipette, and the container was covered with a lid and shaken for stirring for 30 seconds by hand. Thereafter, the lid of the container was removed and the container was connected to a tube. The tube had cocks mounted at both ends thereof, and the cock on the side of the first plug was removed to connect the container to the tube.

Here, the first, third, fifth, and seventh plugs were formed of silicone oils. A first washing liquid of the second plug was an aqueous solution of 76 mass % of guanidinium hydrochloride. A second washing liquid of the sixth plug was a tris-hydrochloric acid buffer solution (solute concentration: 5 mM) of which the pH was 8.0. An eluate of the fourth plug was sterilized water.

The magnetic beads in the container were introduced into the tube by moving a permanent magnet by hand. The magnetic beads were moved up to the fourth plug. Times for which the magnetic beads existed in the respective plugs in the tube were roughly as follows: first, third and seventh plugs: 3 seconds; second plug: 20 seconds; sixth plug: 20 seconds; and fourth plug: 30 seconds. An operation of vibrating the magnetic beads was not performed in the second and sixth plugs. The volumes of the second, sixth, and fourth plugs were 25 μL, 25 μL, and 1 μL, respectively.

Next, the cock on the side of the fifth plug of the tube was removed and the container was deformed by hand to discharge the fifth plug and the fourth plug to a reaction container of PCR. This operation was performed after the magnetic beads were moved and retreated up to the second plug using the permanent magnet.

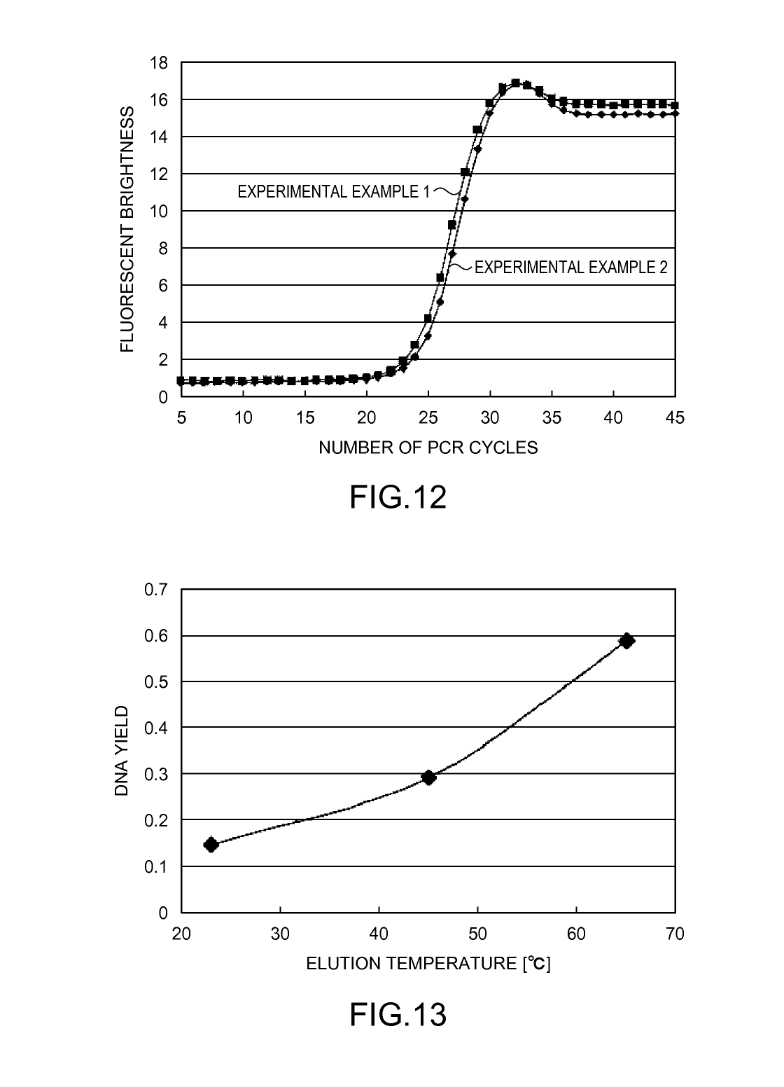

To the extracted liquid, 19 μL of a reaction reagent of PCR was added to perform real-time PCR according to the rule. Details of the reaction reagent of PCR are as follows: 4 μL of Lightcycler 480 genotyping master (manufactured by Roche Diagnostics K.K. 4 707 524); 0.4 μL of SYBR Green I (manufactured by Life Technologies Corporation, S7563) diluted 1,000 times with sterilized water; 0.06 μL of each of primers (F/R) for 100 μM β-actin detection; and 14.48 μL of sterilized water. An amplification curve of PCR of Experimental Example 1 is illustrated in FIG. 12. In FIG. 12, the vertical axis indicates fluorescent brightness and the horizontal axis indicates the number of cycles of PCR.

### 5.2. Experimental Example 2

Nucleic acid extraction was performed using a general nucleic acid extraction method in Experimental Example 2.

First, 375 μL of an adsorbent liquid and 20 μL of a magnetic bead dispersion liquid were accommodated in a polyethylene container having a capacity of 1.5 mL. The compositions of the adsorbent liquid and the magnetic bead dispersion liquid are similar to those of the above-described experimental example.

Next, 50 μL of blood collected from a person was introduced from an inlet of the container using a pipette. The container was covered with a lid to perform stirring for 10 minutes using a vortex mixer, and a magnetic stand and a pipette were operated to perform a B/F separation operation. In this state, the magnetic beads and a small amount of the adsorbent liquid remained in the container.

Next, 450 μL of a first washing liquid having the same composition as that of Experimental Example 1 was introduced to the container. The container was covered with a lid to perform stirring for 5 seconds using the vortex mixer, and the magnetic stand and the pipette were operated to remove the first washing liquid. This operation was repeated two times. In this state, the magnetic beads and a small amount of the first washing liquid remained in the container.

Next, 450 μL of a second washing liquid having the same composition as that of Experimental Example 1 was introduced to the container. The container was covered with a lid to perform stirring for 5 seconds using the vortex mixer, and the magnetic stand and the pipette were operated to remove the second washing liquid. This operation was repeated two times. In this state, the magnetic beads and a small amount of the second washing liquid remained in the container.

In addition, 50 μL of sterilized water (eluate) was added to the container. The container was covered with a lid to perform stirring for 10 minutes using the vortex mixer, and the magnetic stand and the pipette were operated to recover a supernatant liquid. This supernatant liquid contains a target nucleic acid.

1 μL of the extracted liquid was dispensed and 19 of a reaction reagent of PCR was further added thereto to perform real-time PCR according to the rule. Details of the reaction reagent of PCR are as follows: 4 μL of Lightcycler 480 genotyping master (manufactured by Roche Diagnostics K.K. 4 707 524); 0.4 μL of SYBR Green I (manufactured by Life Technologies Corporation, S7563) diluted 1,000 times with sterilized water; 0.06 μL of each of primers (F/R) for 100 μM β-actin detection; and 14.48 μL of sterilized water. An amplification curve at this time is illustrated in FIG. 12.

### 5.3. Experimental Example 3

Experimental Example 3 used a configuration in which the plugs ranging from the first plug 10 to the fifth plug 50 are provided in the tube 200 in the above-described nucleic acid extraction kit 2000.

The composition of an adsorbent liquid and a magnetic bead dispersion liquid were similar to those of Experimental Example 1, and the first, third, and fifth plugs were formed of silicone oils as in Experimental Example 1.

A first washing liquid of the second plug was a tris-hydrochloric acid buffer solution (solute concentration: 5 mM) of which the pH was 8.0. An eluate of the fourth plug was sterilized water.

50 μL of blood collected from a person was put from an inlet of a container using a pipette, and the container was covered with a lid and shaken for stirring for 30 seconds by hand. Thereafter, the lid of the container was removed and the container was connected to a tube. The tube had cocks mounted at both ends thereof, and the cock on the side of the first plug was removed to connect the container to the tube.

The magnetic beads in the container were introduced into the tube by moving a permanent magnet by hand. The magnetic beads were moved up to the fourth plug. Times for which the magnetic beads existed in the respective plugs in the tube were roughly as follows: first and third plugs: 3 seconds; second plug: 20 seconds; and fourth plug: 30 seconds. An operation of vibrating the magnetic beads was not performed in the second plug. The volumes of the second and fourth plugs were 25 μL and 1 μL, respectively.

Next, the cock on the side of the fifth plug of the tube was removed and the container was deformed by hand to discharge the fifth plug and the fourth plug to a reaction container of PCR. This operation was performed after the magnetic beads were moved and retreated up to the second plug using the permanent magnet.

To the extracted liquid, 19 μL of a reaction reagent of PCR was added to perform real-time PCR according to the rule. Details of the reaction reagent of PCR are as follows: 4 μL of Lightcycler 480 genotyping master (manufactured by Roche Diagnostics K.K. 4 707 524); 0.4 μL of SYBR Green I (manufactured by Life Technologies Corporation, S7563) diluted 1,000 times with sterilized water; 0.06 μL of each of primers (F/R) for 100 μM β-actin detection; and 14.48 μL of sterilized water.

An amplification curve at this time had approximately the same features as FIG. 12. In this experimental example, when 76 mass % of guanidinium hydrochloride was used as the first washing liquid of the second plug to perform a similar experiment, a delay in rise of ten cycles or more was shown compared to the amplification curve of Experimental Example 1.

### 5.4. Experimental Example 4

### Influence of Elution Temperature on DNA Yield

Nucleic acid extraction was performed using a general nucleic acid extraction method in Experimental Example 4.

First, 375 μL of an adsorbent liquid and 20 μL of a magnetic bead dispersion liquid were accommodated in a polyethylene container having a capacity of 1.5 mL. The compositions of the adsorbent liquid and the magnetic bead dispersion liquid are similar to those of the above-described Experimental Example 1.

Next, 50 μL of a genomic DNA solution having a concentration adjusted to 1 ng/μL was introduced from an inlet of the container using a pipette. The container was covered with a lid to perform stirring for 10 minutes using a vortex mixer, and a magnetic stand and a pipette were operated to perform a B/F separation operation. In this state, the magnetic beads and a small amount of the adsorbent liquid remained in the container.

Next, 450 μL of a first washing liquid having the same composition as that of Experimental Example 1 was introduced to the container. The container was covered with a lid to perform stirring for 5 seconds using the vortex mixer, and the magnetic stand and the pipette were operated to remove the first washing liquid. This operation was repeated two times. In this state, the magnetic beads and a small amount of the first washing liquid remained in the container.

Next, 450 μL of a second washing liquid having the same composition as that of Experimental Example 1 was introduced to the container. The container was covered with a lid to perform stirring for 5 seconds using the vortex mixer, and the magnetic stand and the pipette were operated to remove the second washing liquid. This operation was repeated two times. In this state, the magnetic beads and a small amount of the second washing liquid remained in the container.

In addition, 50 μL of sterilized water (eluate) was added to the container. The container was covered with a lid to perform stirring for 5 seconds using the vortex mixer, and then heated for 2 minutes using a tube heater. Thereafter, stirring was performed again for 10 seconds using the vortex mixer, and the magnetic stand and the pipette were operated to recover a supernatant liquid. At this time, the heating temperature in the tube heater was set to three stages of 23° C. (leaving at room temperature), 45° C., and 65° C.

1 μL of the extracted liquid was dispensed and 19 μL of a reaction reagent of PCR was further added thereto to perform real-time PCR according to the rule. At this time, as a comparative sample, a genomic DNA solution having a concentration adjusted to 1 ng/1 μL was also added to the PCR reaction sample. Details of the reaction reagent of PCR are as follows: 4 μL of Lightcycler 480 genotyping master (manufactured by Roche Diagnostics K.K. 4 707 524); 0.4 μL of SYBR Green I (manufactured by Life Technologies Corporation, S7563) diluted 1,000 times with sterilized water; 0.06 μL of each of primers (F/R) for 100 μM β-actin detection; and 14.48 of sterilized water.

The relation between an elution temperature and a DNA yield at this time is illustrated in FIG. 13. This result was obtained through calculation from a rise cycle of the real-time PCR. When a rise cycle of the comparative sample is represented by Ct0 and a rise cycle of the extracted sample is represented by Ct1, the DNA yield is expressed as the expression “2^{(Ct0-Ct1)}” as a ratio of the comparative sample (set to 1).

### 5.5. Experimental Example 5

In Experimental Example 5, in a tube coated with a substance having an antistatic effect, an effect of suppressing positional deviation and division of a plug in the tube was examined.

First, twenty polypropylene tubes having an internal diameter of 1 mm were prepared.

Among these, ten tubes were subjected to coating with a modified silicone oil (manufactured by Shin-Etsu Chemical Co., Ltd., X-22-160AS) having an antistatic effect on their outer surfaces. Each of the tubes was filled with a silicone oil having a kinematic viscosity of 2 cSt (25° C.) as a first plug and 1 μl of pure water as a second plug.

Similarly, each of other ten tubes was also filled with a silicone oil and pure water, but their outer surfaces were not coated with a modified silicone oil.

Next, the part filled with the silicone oil and the water in each tube was held by a hand wearing a nitrile glove, and the hand was vertically moved to reciprocate ten times. The number of tubes in which the position of a liquid surface of the second plug was deviated by 1 mm or greater, and the number of tubes in which the second plug was divided were counted.

As a result, the ten tubes subjected to coating with the modified silicone oil on their outer surfaces had no positional deviation of the second plug and no division of the second plug. In contrast, eight of the ten tubes which had not been subjected to coating with the modified silicone oil on their outer surfaces had positional deviation of the liquid surface of the second plug or division of the second plug.

### 5.6. Experimental Example 6

In Experimental Example 6, in a tube having an antistatic film wound thereon, an effect of suppressing positional deviation and division of a second plug in the tube was examined.

This experimental example was carried out in the same manner as in Experimental Example 5, except that in place of coating of the tube with a modified silicone oil as an antistatic agent, Seiden Crystal (manufactured by Achilles corporation) as an antistatic film was wound on the tube.

As a result, the ten tubes having the antistatic film wound thereon had no positional deviation of the liquid surface of the second plug and no division of the second plug. In contrast, eight of the ten tubes having no antistatic film wound thereon had positional deviation or division of the second plug.

### 5.7. Experimental Example 7

In Experimental Example 7, in an antistatic tube, an effect of suppressing positional deviation and division of a second plug in the tube was examined.

In this experimental example, ten antistatic tubes (manufactured by CKD) having an internal diameter of 1.8 mm and ten polypropylene tubes having an internal diameter of 1.8 mm were prepared.

Each of the tubes was filled with a silicone oil having a kinematic viscosity of 2 cSt (25° C.) as a first plug and 1 μl of pure water as a second plug.

Next, the part filled with the silicone oil and the water in each tube was held by a hand wearing a nitrile glove, and the hand was vertically moved to reciprocate ten times. The number of tubes in which the position of a liquid surface of the second plug was deviated by 1 mm or greater, and the number of tubes in which the second plug was divided were counted.

As a result, the ten antistatic tubes had no positional deviation and division of the second plug. In contrast, nine of the ten polypropylene tubes had positional deviation of the liquid surface of the second plug or division of the second plug.

### 5.8. Experimental Example 8

In Experimental Example 8, using silicone oils containing various antistatic agents added thereto as oil plugs, an effect of suppressing positional deviation and division of a second plug in a tube were examined to examine a volume specific resistivity with which the suppression effect was high.

This experimental example was carried out in the same manner as in Experimental Example 5, except that silicone oils containing the various antistatic agents shown in Table 1 added thereto were used as oil plugs in polypropylene tubes having an internal diameter of 1 mm. Ten tubes were prepared for each condition.

Next, the plug part filled with the silicone oil in each tube was held by a hand wearing a nitrile glove, and the hand was vertically moved to reciprocate ten times. The number of tubes in which the plug was divided or the position of a liquid surface of the plug was moved by 1 mm or greater was counted, and the oil with no division or movement of the second plug in eight or more of the ten tubes was indicated by “O” in Table 1.

Volume specific resistivities of the silicone oils containing the various additives were measured. The measurement was performed at a measurement voltage of 10 V using Universal Electrometer (manufactured by Kawaguchi Electric Works, MMA-II-17B). The results are shown in Table 1.

TABLE 1

| | | | Amount | | Volume |
|---|---|---|---|---|---|
| | | | to be | | Specific |
| Product | | | Added | Movement | Resistivity |
| Name | Maker | Component Name | (%) | of Plug | (Ω·cm) |
| X-22-160AS | Shin-Etsu | Carbinol-Modified Silicone | 5 | X | 4.8 × 10¹³ |
| | Chemical Co., | Oil | 4 | X | 4.0 × 10¹³ |
| | Ltd. | | | | |
| XS-66-B8226 | Momentive | Trifluoroalkyldimethyl | 4 | ◯ | 5.4 × 10¹⁰ |
| | Performance | Trimethylsiloxysilicic Acid | 0.005 | X | 9.0 × 10¹² |
| | Materials | | | | |
| | Japan LLC. | | | | |
| XS-66-C1191 | Momentive | Trifluoroalkyldimethyl | 4 | X | 7.8 × 10¹⁰ |
| | Performance | Trimethylsiloxysilicic Acid | 0.005 | X | 1.1 × 10¹² |
| | Materials | | | | |
| | Japan LLC. | | | | |
| X21-5250 | Shin-Etsu | 50% Trimethylsiloxysilicic | 4 | X | 3.6 × 10¹¹ |
| | Chemical Co., | Acid, 50% Cyclopentasiloxane | 0.5 | X | 8.0 × 10¹² |
| | Ltd. | | | | |
| SilFortm | Momentive | Polymethylsilsesquioxane | 1 | X | 9.0 × 10¹² |
| Flexible | Performance | | | | |
| Resin | Materials | | | | |
| | Japan LLC. | | | | |

### 5.5. Experiment Results

The following facts were found from the above-described experimental examples.

(1) In comparison in terms of the time required for the nucleic acid extraction process as preprocessing of PCR, the time from when the sample was inserted into the container to when the target nucleic acid was introduced to the reaction container of PCR was approximately 2 minutes in Experimental Example 1, and was approximately 30 minutes in Experimental Example 2. Accordingly, it was found that the time required for nucleic acid extraction was significantly reduced when using the nucleic acid extraction method of Experimental Example 1, as opposed to the nucleic acid extraction method of Experimental Example 2.

(2) The amount of each washing liquid in Experimental Example 1 was approximately 1/18 of the amount of each washing liquid in Experimental Example 2. The amount of the eluate in Experimental Example 1 was approximately 1/50 of the amount of the eluate in Experimental Example 2. Accordingly, it was found that in Experimental Example 1, extremely smaller amounts of the washing liquid and the eluate suffice compared to Experimental Example 2.

(3) In comparison in terms of the concentration of the target nucleic acid in the eluate using the amounts of the adsorbent liquid and the eluate, ideally, Experimental Example 1 is thought to be 50 times higher than Experimental Example 2 in concentration. However, in the present experimental example, the amount of the nucleic acid contained in the blood sample is large and exceeds the adsorbency amount of 1 μL of the magnetic beads, and the entire amount of the nucleic acid contained in the blood sample cannot be recovered, whereby a concentration 50 times higher than the concentration of Experimental Example 2 is not obtained in Experimental Example 1. In the case of a sample in which the amount of the nucleic acid contained is small and does not exceed the adsorbency amount of 1 μL of the magnetic beads, a concentration 50 times higher than the concentration of Experimental Example 2 is obtained in Experimental Example 1.

(4) When viewing the graphs of FIG. 12, it was found that the rise of the amplification factor of the nucleic acid is shown earlier in Experimental Example 1 than in Experimental Example 2 by approximately 0.6 cycles. That is, it was found that the reaction liquid of PCR used in Experimental Example 1 is higher than the reaction liquid of PCR used in Experimental Example 2 in concentration of the target nucleic acid. Accordingly, the fact that Experimental Example 1 is higher than Experimental Example 2 in concentration of the target nucleic acid in the eluate was supported.

(5) From the results of Experimental Example 3, it was found that even when the second plug is a buffer, sufficient extraction is possible. It was also found that when the second plug is an aqueous solution of guanidine, the rise of the PCR amplification curve is significantly delayed due to the influence of enzyme reaction inhibition. It was also found that when diluting the extracted liquid at least 1,000 times, it is possible to suppress the influence of enzyme reaction inhibition of the aqueous solution of guanidine.

(6) From the results of Experimental Example 4, it was found that when the fourth plug is heated higher than approximately 40° C., the DNA yield suffices for being used in PCR.

(7) From the results of Experimental Examples 5 to 7, it was found that when electrification of the tube is prevented, it is possible to suppress the electric interaction of the aqueous plug with the tube wall. From the results of Experimental Example 8, it was found that when using an oil plug having a volume specific resistivity of 5.4×10¹⁰ Ω·cm or lower, the suppression effect is high.

The invention is not limited to the above-described embodiments, and various modifications can be made. For example, the invention includes configurations substantially the same as those described in the embodiments (for example, configurations having substantially the same functions, methods, and results, and configurations having substantially the same objects and effects). The invention also includes configurations in which non-essential parts of the configurations described in the embodiments are replaced with others. The invention also includes configurations that achieve the same advantages or achieve the same objects as those of the configurations described in the embodiments. The invention also includes configurations in which known techniques are added to the configurations described in the embodiments.

The entire disclosure of Japanese Patent Application No. 2013-213466, filed Oct. 11, 2013 is expressly incorporated by reference herein.

What is claimed is:

1. A nucleic acid extraction device comprising:
a tube in which a first plug formed of an oil, a second plug formed of a washing liquid which does not mix with an oil, a third plug formed of an oil, a fourth plug formed of an eluate which does not mix with an oil, and a fifth plug formed of an oil are arranged in this order,
wherein the tube is subjected to an antistatic treatment.

2. The nucleic acid extraction device according to claim 1,
wherein the tube is coated with an antistatic agent.

3. The nucleic acid extraction device according to claim 1,
wherein an antistatic sheet is wound on the tube.

4. The nucleic acid extraction device according to claim 1,
wherein the tube includes an antistatic material.

5. The nucleic acid extraction device according to claim 1,
wherein the first plug, the third plug, or the fifth plug contains an antistatic agent.

6. The nucleic acid extraction device according to claim 5,
wherein the antistatic agent has a volume specific resistivity of 5.4×10¹⁰ Ω·cm or lower.

7. A nucleic acid extraction apparatus comprising:
the nucleic acid extraction device according to claim 1;
a magnetic force application portion which applies, when the tube is mounted on a mounting portion, a magnetic force from a side surface of the tube; and
a moving mechanism which changes the relative arrangement between the mounting portion and the magnetic force application portion in a longitudinal direction of the tube.

8. A nucleic acid extraction apparatus comprising:
the nucleic acid extraction device according to claim 2;
a magnetic force application portion which applies, when the tube is mounted on a mounting portion, a magnetic force from a side surface of the tube; and
a moving mechanism which changes the relative arrangement between the mounting portion and the magnetic force application portion in a longitudinal direction of the tube.

9. A nucleic acid extraction apparatus comprising:
the nucleic acid extraction device according to claim 3;
a magnetic force application portion which applies, when the tube is mounted on a mounting portion, a magnetic force from a side surface of the tube; and
a moving mechanism which changes the relative arrangement between the mounting portion and the magnetic force application portion in a longitudinal direction of the tube.

10. A nucleic acid extraction kit comprising:
the nucleic acid extraction device according to claim 1; and
a container which can be connected to an end on the side of the first plug of the tube by internal communication.

11. A nucleic acid extraction kit comprising:
the nucleic acid extraction device according to claim 2; and
a container which can be connected to an end on the side of the first plug of the tube by internal communication.

12. A nucleic acid extraction kit comprising:
the nucleic acid extraction device according to claim 3; and
a container which can be connected to an end on the side of the first plug of the tube by internal communication.

13. A nucleic acid extraction device comprising:
a tube in which a first plug formed of an oil and a second plug formed of an aqueous solution which does not mix with the oil are arranged,
wherein the tube is subjected to an antistatic treatment.

14. The nucleic acid extraction device according to claim 13,
wherein the tube is coated with an antistatic agent.

15. The nucleic acid extraction device according to claim 13,
wherein an antistatic sheet is wound on the tube.

16. The nucleic acid extraction device according to claim 13,
wherein the tube includes an antistatic material.

17. The nucleic acid extraction device according to claim 13,
wherein the oil contains an antistatic agent.

18. The nucleic acid extraction device according to claim 17,
wherein the antistatic agent has a volume specific resistivity of 5.4×10¹⁰ Ω·cm or lower.

19. A nucleic acid extraction apparatus comprising:
the nucleic acid extraction device according to claim 13;
a magnetic force application portion which applies, when the tube is mounted on a mounting portion, a magnetic force from a side surface of the tube; and
a moving mechanism which changes the relative arrangement between the mounting portion and the magnetic force application portion in a longitudinal direction of the tube.

20. A nucleic acid extraction apparatus comprising:
the nucleic acid extraction device according to claim 14;
a magnetic force application portion which applies, when the tube is mounted on a mounting portion, a magnetic force from a side surface of the tube; and
a moving mechanism which changes the relative arrangement between the mounting portion and the magnetic force application portion in a longitudinal direction of the tube.
